(19) United States
(12) Patent Application Publication
(10) Pub. No.: US 2013/0122484 A1
(43) Pub. Date: May 16, 2013

(54) DIAGNOSTIC METHOD FOR DETERMINING ANIMALS PERSISTENTLY INFECTED (PI) WITH BOVINE VIRAL DIARRHEA VIRUS (BVDV)

(71) Applicants: John EL-ATTRACHE, Lenexa, KS (US); Quoc HOANG, Austin, TX (US); Catherine O'CONNELL, Austin, TX (US)

(72) Inventors: John EL-ATTRACHE, Lenexa, KS (US); Quoc HOANG, Austin, TX (US); Catherine O'CONNELL, Austin, TX (US)

(73) Assignee: LIFE TECHNOLOGIES CORPORATION, Carlsbad, CA (US)

(21) Appl. No.: 13/633,068

(22) Filed: Oct. 1, 2012

Related U.S. Application Data
(60) Provisional application No. 61/542,171, filed on Oct. 1, 2011.

(51) Int. Cl. C12Q 1/70

(52) U.S. Cl. CPC C12Q 1/70
USPC 435/5

(57) ABSTRACT

The present specification relates to methods and kits for detection of animals that are persistently infected (PI) with a Bovine Viral Diarrhea Virus (BVDV) and/or transiently infected (TI) with BVDV. Some embodiments describe methods to distinguish a PI animal from a TI animal using a single one-time testing protocol.

### CROSS-REFERENCE TO RELATED APPLICATION

This application claims priority to U.S. Provisional Application Ser. 61/542,171, entitled “Diagnostic Method for Determining Animals Persistently Infected with Bovine Viral Diarrhea Virus (BVDV)”, filed Oct. 1, 2011, and the entire content of which is incorporated herein by reference.

### FIELD

The present specification relates to methods and kits for detection of animals (e.g., such as but not limited to cattle, sheep, goats, deer, alpacas) that are persistently infected (PI) with a Bovine Viral Diarrhea Virus (BVDV) and/or animals that are transiently infected (TI) with BVDV. The present disclosure also provides methods to distinguish a PI BVDV animal from a TI BVDV animal using a single-one time test.

### BACKGROUND

Bovine Viral Diarrhea Virus (BVDV) is a pestivirus that infects cattle and a number of other mammals. BVDV is a cause of major economic loss in both the beef and dairy industries, estimated to be ˜$2-3 billion annually in the US alone. BVDV can infect cattle and cause an animal to be either Persistently Infected (PI) or Transiently Infected (TI).

A PI infection is caused by exposure of bovine fetus to BVDV between 40 to 125 days of gestation leading to life-long immunotolerance of calf. Thus, PI animals are the primary reservoir of BVDV infection in herds. A TI infection is caused in animals that are exposed to a BVD virus after birth. TI animals generally show clinical signs of infection and usually pass the infection within 3 weeks after exposure.

Current methods for determination of PI animals requires two positive results from the same animal taken at least 3 weeks apart. This ensures the distinction between PI and TI animals. Methods of BVDV testing may involve Antigen Capture ELISA (ACE), Immunohistochemistry (IHC), Virus Isolation (VI) and qRT-PCR. However, irrespective of the method, two tests at least 3 weeks apart are required. There is a need for a better, faster and more economical method for discriminating between PI and TI animals.

### SUMMARY

The present specification relates in some embodiments to methods and kits for detection of PI and/or TI animals infected with a Bovine Viral Diarrhea Virus (BVDV) and methods of distinguishing PI animals from TI animals. In some embodiments, methods according to the present disclosure comprise a one-step detection which is rapid and offers the advantage of not having to re-test animals to determine PI vs TI infection.

In some embodiments, a method of the disclosure for diagnosing an animal as persistently infected (PI) with a bovine viral diarrhea virus (BVDV) comprises: a) performing a single BVDV polymerase chain reaction (PCR) test comprising: 1) contacting nucleic acids from a sample derived from an animal with at least one primer set that is specific to hybridize to one or more BVDV-specific target nucleic acids; 2) performing an PCR amplification on nucleic acids from the sample hybridized to the at least one primer set; and 3) detecting one or more BVDV-specific amplification products; and b) determining Ct value of the BVDV-specific amplification product, wherein a Ct value of ≦31 indicates that the animal is a PI animal. In some embodiments of this method, a Ct value in the range of from about 20 about 31 indicates an animal is a PI animal.

A method of the disclosure, in one embodiment, is a method to detect a TI animal and comprises: a) performing a single BVDV polymerase chain reaction (PCR) test comprising: 1) contacting nucleic acids from a sample derived from an animal with at least one primer set that is specific to hybridize to one or more BVDV-specific target nucleic acids; 2) performing an PCR amplification on nucleic acids from the sample hybridized to the at least one primer set; and 3) detecting one or more BVDV-specific amplification products; and b) determining Ct value of the BVDV-specific amplification product, wherein a Ct value of >31 indicates that the animal is a TI animal. In some embodiments of this method, a Ct value having a range of values of from about 32 to about 40 is indicative of a TI animal.

Some embodiments of the present disclosure describe a method to distinguish an animal with a BVDV infection as a PI animal or a TI animal comprising: performing a PCR reaction to amplify and detect the presence of one or more BVDV-specific target nucleic acids in a sample obtained from the animal; and determining Ct value of one or more BVDV-specific target nucleic acid amplification products, wherein a Ct value of >31 indicates that the animal is a TI animal and a Ct value ≦31 indicates that the animal is a PI animal. In some embodiments, a method of the disclosure can be used to determine if a BVDV infection in an animal is a PI or a TI BVDV infection.

In some embodiments, determining a Ct value comprises calculating the BVDV titer in a sample.

In the methods described above, nucleic acids from an animal derived sample can comprise DNA, RNA, genomic DNA and combinations thereof. In embodiments where a nucleic acids from the sample comprises a BVDV RNA molecule, a method of the disclosure can further comprises performing an reverse transcriptase reaction on nucleic acids from the sample prior to performing the PCR test. A reverse transcriptase (RT) reaction converts a BVDV-specific RNA molecule into a cDNA molecule and a PCR reaction can then be performed on the BVDV-specific cDNA molecule using one or more BVDV-specific primer set(s).

In some embodiments, nucleic acids can extracted and/or isolated and/or purified from a sample obtained from an animal suspected of being infected with BVDV. Several methods known in the art may be used for extraction/isolation of nucleic acids from a variety of animal derived samples.

Animals that can be tested and/or diagnosed by a method include any mammal that can be afflicted with a BVDV. Non limiting examples include animals such as cattle, sheep, goats, deer and/or alpacas.

A sample derived/obtained from an animal to be tested for BVDB can be an ear notch sample, a hair sample, a tail hair sample, a serum sample, a blood sample, a milk sample, a urine sample, a fecal sample, a skin sample, a lymph sample, a plasma sample, a cerebrospinal fluid sample, a mucus sample, a throat swab sample, and/or any sample from a bodily fluid or tissue from an animal suspected of having BVDV. In some embodiments, ear notch, skin, hair and milk samples are preferable.

Methods of the disclosure are applicable for detecting/diagnosing/distinguishing PI and TI animals regardless of the BVDV sub-type afflicting the animal. For example, methods described here can be used to detect various BVDV sub-types for PI or TI infection, such as, but not limited to, a BVDV subtype 1A, a BVDV subtype 1B and/or a BVDV subtype 2A.

In some embodiments, kits for detecting BVDV, according to the disclosure may provide instructions to test for Ct values as described or Ct ranges as described to make a diagnosis of TI or PI in BVDV animals. In some embodiments, kits of the disclosure can have software components that can calculate the Ct value or Ct range and output the results as PI or TI positive (or negative) on a screen or in a printout. Software programs can be part of RT-PCR or PCR machines and can comprise a GUI for user friendly steps for calculations and analysis.

Some embodiments of the present disclosure may provide one or more technical advantages. Methods of the disclosure, in some embodiments, advantageously reduce the time needed for testing by eliminating the need for a second test post three weeks of the first test to confirm the PI/TI diagnosis of an animal. Methods of the disclosure, in some embodiments, advantageously reduce the time and resources needed for multiple testing.

Methods of the disclosure can be performed on automated platforms, semi-automated platforms and/or manually.

While specific advantages have been disclosed hereinabove, it will be understood that various embodiments may include all, some, or none of the previously disclosed advantages. Other technical advantages may become readily apparent to those skilled in the art in light of the teachings of the present disclosure. These and other features of the present teachings will become more apparent from the detailed description in sections below.

### BRIEF DESCRIPTION OF THE DRAWINGS

One or more embodiments of the present disclosure may be better understood in reference to one or more the drawings below. The skilled artisan will understand that the drawings, described below, are for illustration purposes only. The drawings are not intended to limit the scope of the present teachings in any way.

FIG. 1 depicts a BVDV testing workflow, according to one embodiment of the disclosure;

FIG. 2 depicts Ct values for sixty three confirmed PI samples, according to one embodiment of the disclosure;

FIG. 3 depicts no statistical differences in Ct values for cattle afflicted with different BVDV subtypes, according to one embodiment of the disclosure;

FIG. 4 depicts Ct values in PI animal samples at day of arrival and at day 27, according to one embodiment of the disclosure;

FIG. 5 depicts Ct values for TI animals, according to one embodiment of the disclosure;

FIG. 6 depicts a histogram showing Ct values and ranges of Ct values in PI and TI animals, according to one embodiment of the disclosure;

FIG. 7 depicts Ct values for PI and TI animals, according to one embodiment of the disclosure;

FIG. 8 depicts Kernel density of PI subpopulations, according to one embodiment of the disclosure; and

FIG. 9 describes Tables A-F showing data of BVDV testing.

### DETAILED DESCRIPTION

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range therebetween unless otherwise specifically stated. For example, “from 0.2 to 0.5” means 0.2, 0.3, 0.4, 0.5; ranges therebetween such as 0.2-0.3, 0.3-0.4, 0.2-0.4; increments there between such as 0.25, 0.35, 0.225, 0.335, 0.49; increment ranges there between such as 0.26-0.39; and the like.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The term “extraction” and “extracted” refers to the act or process of removing or isolating a substance (e.g., a biomolecule such as DNA, RNA, protein) from a mixture (of several components such as cellular components, materials in a sample in which the cell that has the biomolecule is comprised in). An extracted substance may have significantly decreased quantities of components that it was present in, may be substantially pure or may be pure (devoid of any contaminants). “Substantially pure” or “pure” substances may be also referred to as “purified” by “purification” methods or steps.

The term “cells” refers to the smallest structural unit of an organism that is capable of independent functioning, consisting of one or more nuclei, cytoplasm, and various organelles, all surrounded by a semipermeable cell membrane.

The terms “ambient conditions” and “room temperature” are interchangeable and refer to common, prevailing, and uncontrolled atmospheric and weather conditions in a room or place.

“Hybridization” refers to a process in which single-stranded nucleic acids with complementary or near-complementary base sequences interact to form hydrogen-bonded complexes called hybrids. Hybridization reactions are sensitive and selective. In vitro, the specificity of hybridization (i.e., stringency) is controlled by factors such as the concentrations of salt or formamide in prehybridization and hybridization solutions and by the hybridization temperature. In some embodiments, stringency may be increased by reducing the concentration of salt, increasing the concentration of formamide, and/or by raising the hybridization temperature. For example, high stringency conditions could occur at about 50% formamide at 37° C. to 42° C. Reduced stringency conditions could occur at about 35% to 25% formamide at 30° C. to 35° C. Some examples of stringency conditions for hybridization are also described in Sambrook, J., 1989, Molecular Cloning A Laboratory Manual, Cold Spring Harbor Laboratory Press, Cold Spring Harbor, N.Y. Generally, the temperature for hybridization is about 5-10° C. less than the melting temperature (Tm) of a hybrid nucleic acid.

As used herein, the phrase “stringent hybridization conditions” refers to hybridization conditions which can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically “substantially complementary” to a second polynucleotide if hybridization occurs between the polynucleotide and the second polynucleotide. As used herein, “specific hybridization” or “oligo (primer/probe) specific to hybridize to” refers to hybridization between two polynucleotides under stringent hybridization conditions.

As used herein, the phrase “nucleic acid molecule” refers to a sequence of contiguous nucleotides (riboNTPs, dNTPs or ddNTPs, or combinations thereof) of any length which can encode a full length polypeptide or a fragment of any length thereof, or which can be non-coding. The term “oligonucleotide” refers to a synthetic or natural molecule comprising a covalently linked sequence of nucleotides which are joined by a phosphodiester bond between the 3′ position of the pentose of one nucleotide and the 5′ position of the pentose of the adjacent nucleotide. As used herein, the phrase “nucleic acid,” “nucleic acid sequence,” “oligonucleotide”, and “polynucleotides” are interchangeable and not intended to be limiting. As used herein, the term “polynucleotide” refers to a polymeric form of nucleotides of any length, either ribonucleotides, deoxynucleotides, or peptide nucleic acids (PNA), and includes both double- and single-stranded RNA, DNA, and PNA. A polynucleotide may include nucleotide sequences having different functions, including, for instance, coding regions, and non-coding regions such as regulatory regions. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. An “oligonucleotide” can in some embodiments refer to a polynucleotide such as a primer and/or a probe.

As used herein a “target-specific polynucleotide” or “targets-specific nucleic acids” refers to an oligonucleotide or a polynucleotide having a target-binding segment that is perfectly or substantially complementary to a target sequence, such that the polynucleotide binds specifically to an intended target without significant binding to non-target sequences under sufficiently stringent hybridization conditions. The target-specific polynucleotide can be e.g., a primer or probe and the subject of hybridization with its complementary target sequence.

The term “target sequence”, “target signature sequence” “target nucleic acid”, “target” or “target polynucleotide sequence” refers to a nucleic acid of interest. Example targets of interest in some embodiments of this application include nucleic acids that are uniquely present in a BVDV organism and fragments, complements and sequences with 90% homology thereto. A “BVDV target sequence” or “BVDV-specific target nucleic acid sequence” can be a polynucleotide sequence that is the subject of hybridization with a complementary polynucleotide, e.g. a primer or probe. A target sequence may be known or not known, in terms of its actual sequence, however its amplification can be used to determine the presence of the organism it belongs (e.g., a BVDV organism in this application). The target sequence may or may not be of biological significance. As non-limiting examples, target sequences may include regions of genomic DNA, regions of genomic DNA which are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc.

As used herein an “amplified target polynucleotide sequence product” refers to the resulting amplicon from an amplification reaction such as a polymerase chain reaction. The resulting amplicon product arises from hybridization of complementary primers to a target polynucleotide sequence under suitable hybridization conditions and the repeating in a cyclic manner the polymerase chain reaction as catalyzed by DNA polymerase for DNA amplification or RNA polymerase for RNA amplification.

As used herein, the “polymerase chain reaction” or PCR is a an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art.

As used herein, “amplification” or “amplify” and the like refers to a process that results in an increase in the copy number of a molecule or set of related molecules. As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods may comprise thermal-cycling or may be performed isothermally. In various embodiments, the term “amplification product” or “amplified product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated. According to certain embodiments, following at least one amplification cycle, “amplification products” can be detected by several methods such as, but not limited to, separation based on their molecular weight or length or mobility and by sequencing.

The term “end-point” measurement refers to a method where data collection occurs only once the reaction has been stopped. The term “real-time” and “real-time continuous” are interchangeable and refer to a method where data collection occurs through periodic monitoring during the course of the polymerization reaction. Thus, the methods combine amplification and detection into a single step.

Quantitative PCR (QPCR) methods can be used to isolate RNA for quantitative PCR—more specifically, quantitative RT-PCR—to quantitate the RNA in a sample (for e.g. to quantitate RNA in a 500 μl food sample). QPCR methods may be semi-quantitative or fully quantitative.

Two approaches, competitive quantitative PCR™ and real-time quantitative PCR™, both estimate target gene concentration in a sample by comparison with standard curves constructed from amplifications of serial dilutions of standard RNA. However, they differ substantially in how these standard curves are generated. In competitive QPCR, an internal competitor RNA is added at a known concentration to both serially diluted standard samples and unknown (environmental) samples. After coamplification, ratios of the internal competitor and target PCR™ products are calculated for both standard dilutions and unknown samples, and a standard curve is constructed that plots competitor-target PCR™ product ratios against the initial RNA concentration of the standard dilutions. Given equal amplification efficiency of competitor and RNA, the concentration of the latter in environmental samples can be extrapolated from this standard curve.

In real-time QPCR, the accumulation of amplification product is measured continuously in both standard dilutions of RNA and samples containing unknown amounts of RNA. A standard curve is constructed by correlating initial template concentration in the standard samples with the number of PCR™ cycles (Cₜ) necessary to produce a specific threshold concentration of product. In the test samples, a target PCR™ product accumulation is measured after the same Cₜ, which allows interpolation of target RNA concentration from the standard curve. Although real-time QPCR permits more rapid and facile measurement of RNA during routine analyses, competitive QPCR remains an important alternative for quantification in environmental samples. Co-amplification of a known amount of competitor RNA with target RNA is an intuitive way to correct for sample-to-sample variation of amplification efficiency due to the presence of inhibitory substrates and large amounts of background RNA that are obviously absent from the standard dilutions.

As used herein, the term “Ct” represents the PCR cycle number when a PCR signal is first recorded as statistically significant. The “Ct” or cycle threshold, is the calculated PCR cycle where the cumulative fluorescence of the PCR reaction exceeds the background fluorescence of the reaction and crosses a set threshold as defined by the real-time analysis software.

Another type of QPCR is applied quantitatively PCR™. Often termed “relative quantitative PCR,” this method determines the relative concentrations of specific nucleic acids. In the context of the present dislcosure, RT-PCR is performed on RNA samples isolated from food samples.

In PCR™, the number of molecules of the amplified DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified DNA becomes asymptotic to some fixed value. This is said to be the plateau portion of the curve.

The concentration of the DNA in the linear portion of the PCR™ amplification is directly proportional to the starting concentration of the target before the reaction began. By determining the concentration of the amplified products of the DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues, the relative abundances of the specific mRNA from which the target sequence was derived can be determined for the respective tissues. This direct proportionality between the concentration of the PCR™ products and the relative DNA abundances is only true in the linear range of the PCR™ reaction.

The final concentration of the DNA in the plateau portion of the curve is determined by the availability of reagents in the reaction mix and is independent of the original concentration of target DNA. Therefore, the first condition to be met before the relative abundances of a RNA species can be determined by RT-PCR for a collection of RNA populations is that the concentrations of the amplified PCR™ products are sampled when the PCR™ reactions are in the linear portion of their curves.

The second condition to be met for a quantitative RT-PCR experiment to successfully determine the relative abundances of a particular RNA species is that relative concentrations of the amplifiable cDNAs are normalized to some independent standard. The goal of an RT-PCR experiment is to determine the abundance of a particular RNA species relative to the average abundance of all RNA species in the sample.

Most protocols for competitive PCR™ utilize internal PCR™ standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for samples. When samples are of variable quantity (making normalization problematic), and/or if samples are of variable quality (necessitating the co-amplification of a reliable internal control, preferably of a larger size than the target), the issues in these situations are overcome if the RT-PCR is performed as a relative quantitative RT-PCR with an internal standard in which the internal standard is an amplifiable cDNA fragment that is larger than the target cDNA fragment and in which the abundance of the RNA encoding the internal standard is roughly 5-100 fold higher than the RNA encoding the target. This assay measures relative abundance, not absolute abundance of the respective RNA species.

Other studies may be performed using a more conventional relative quantitative RT-PCR assay with an external standard protocol. These assays sample the PCR™ products in the linear portion of their amplification curves. The number of PCR™ cycles that are optimal for sampling is empirically determined for each target cDNA fragment. In addition, the reverse transcriptase products of each RNA population isolated from the various tissue samples is carefully normalized for equal concentrations of amplifiable cDNAs. Since the assay measures absolute mRNA abundance, these steps insure more reliable results. Absolute RNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR product, display of the product on an electrophoretic gel or another display method becomes less complex, has less background and is easier to interpret.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “surrogate” as used herein means a product that is indicative of presence of another product. For example, an amplification product is a surrogate for a nucleic acid that has been amplified.

As used herein, the term “nucleotide” or “nt” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, 7-deaza-dGTP and 7-deaza-dATP. The term nucleotide as used herein also refers to dideoxyribonucleoside triphosphates (ddNTPs) and their derivatives. Examples of dideoxyribonucleoside triphosphates include, but are not limited to, ddATP, ddCTP, ddGTP, ddITP, and ddTTP.

The term “label” refers to any moiety which can be attached to a molecule and: (i) provides a detectable signal; (ii) interacts with a second label to modify the detectable signal provided by the second label, e.g. FRET; (iii) stabilizes hybridization, i.e. duplex formation; or (iv) provides a capture moiety, i.e. affinity, antibody/antigen, ionic complexation. Labeling can be accomplished using any one of a large number of known techniques employing known labels, linkages, linking groups, reagents, reaction conditions, and analysis and purification methods. Labels include light-emitting compounds which generate a detectable signal by fluorescence, chemiluminescence, or bioluminescence (Kricka, L. in Nonisotopic DNA Probe Techniques (1992), Academic Press, San Diego, pp. 3-28). Another class of labels are hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2.sup.nd Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labelling of PCR probes and primers” (1995) in PCR 2: A Practical Approach, Oxford University Press, Oxford, pp. 39-54).

The terms “annealing” and “hybridization” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “end-point analysis” refers to a method where data collection occurs only when a reaction is substantially complete.

The term “real-time analysis” refers to periodic monitoring during PCR. Certain systems such as the Applied Biosystems 7500 Real-Time PCR System (Applied Biosystems, Foster City, Calif.) conduct monitoring during each thermal cycle at a pre-determined or user-defined point. Real-time analysis of PCR with FRET probes measures fluorescent dye signal changes from cycle-to-cycle, preferably minus any internal control signals.

The term “quenching” refers to a decrease in fluorescence of a first moiety (reporter dye) caused by a second moiety (quencher) regardless of the mechanism.

A “primer,” as used herein, is an oligonucleotide that is complementary to a portion of target polynucleotide and, after hybridization to the target polynucleotide, may serve as a starting-point for an amplification reaction and the synthesis of an amplification product. Primers include, but are not limited to, spanning primers. A “primer pair” refers to two primers that can be used together for an amplification reaction. A “PCR primer” refers to a primer in a set of at least two primers that are capable of exponentially amplifying a target nucleic acid sequence in the polymerase chain reaction.

The term “probe” comprises a polynucleotide that comprises a specific portion designed to hybridize in a sequence-specific manner with a complementary region of a specific nucleic acid sequence, e.g., a target nucleic acid sequence. In certain embodiments, the specific portion of the probe may be specific for a particular sequence, or alternatively, may be degenerate, e.g., specific for a set of sequences. In certain embodiments, the probe is labeled. The probe can be an oligonucleotide that is complementary to at least a portion of an amplification product formed using two primers.

The terms “complement” and “complementary” as used herein, refer to the ability of two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary.

A “label” refers to a moiety attached (covalently or non-covalently), or capable of being attached, to an oligonucleotide, which provides or is capable of providing information about the oligonucleotide (e.g., descriptive or identifying information about the oligonucleotide) or another polynucleotide with which the labeled oligonucleotide interacts (e.g., hybridizes). Labels can be used to provide a detectable (and optionally quantifiable) signal. Labels can also be used to attach an oligonucleotide to a surface.

A “fluorophore” is a moiety that can emit light of a particular wavelength following absorbance of light of shorter wavelength. The wavelength of the light emitted by a particular fluorophore is characteristic of that fluorophore. Thus, a particular fluorophore can be detected by detecting light of an appropriate wavelength following excitation of the fluorophore with light of shorter wavelength.

The term “quencher” as used herein refers to a moiety that absorbs energy emitted from a fluorophore, or otherwise interferes with the ability of the fluorescent dye to emit light. A quencher can re-emit the energy absorbed from a fluorophore in a signal characteristic for that quencher, and thus a quencher can also act as a flourophore (a fluorescent quencher). This phenomenon is generally known as fluorescent resonance energy transfer (FRET). Alternatively, a quencher can dissipate the energy absorbed from a fluorophore as heat (a non-fluorescent quencher).

As used herein the term “sample” refers to a starting material suspected of harboring a particular virus (e.g. a BVDV). Examples of samples that can have a BVDV organism include: samples derived from animals that can be infected by a BVDV microbe, such as, but not limited to, cattle, sheep, goats, deer, alpacas; water samples and environmental samples from environments/water sources near animals that are infected by a BVDV (e.g., soil samples, dirt samples, garbage samples, sewage samples, air samples, water samples, which may be derived from animal dens/farms/barns).

A sample derived from an animal to be tested for BVDB can be an ear notch sample, a hair sample, a tail hair sample, a serum sample, a blood sample, a milk sample, a urine sample, a fecal sample, a skin sample, a lymph sample, a plasma sample, a cerebrospinal fluid sample, a mucus sample, a throat swab sample, and/or any sample from a bodily fluid or tissue from an animal suspected of having BVDV.

A sample may be tested directly, or may be prepared or processed in some manner prior to testing. For example, a sample may be processed to separate and/or lyse viral cells contained therein. Lysed viral cells from a sample may be additionally processed or prepares to separate, isolate and/or extract genetic material from the virus for analysis to detect and/or identify the contaminating virus. In some embodiments described here, as sample may be subject to separation to initially separate a virus of interest from other viruses, microbes and other sample components. Separated virus from samples can also be enriched prior to analysis. Analysis of a sample may include one or more molecular methods. For example, according to some exemplary embodiments of the present disclosure, a sample may be subject to nucleic acid amplification (for example by PCR) using appropriate oligonucleotide primer sets (one or more) that are specific to a BVDV specific target nucleic acid sequence. A primer set specific for a BVDV-specific target nucleic acid can in some embodiments have one or more primers that can hybridize specifically to a BVDB-specific target nucleic acid or a fragment or complement thereof and amplify different BVDV subtypes or serovars under amplification conditions. In some embodiments, multiple primer sets may be used to specifically hybridize to and amplify multiple BVDV specific target nucleic acid sequences. Probes specific to hybridize to a BVBD specific targets may also be employed for amplification, such as for a TaqMan® amplification. Amplification products may then be further subject to testing with specific probes (or reporter probes) to allow detection of viral (BVDV) nucleic acid sequences that have been amplified from the sample. In some embodiments, if a viral nucleic acid sequence is amplified from a sample, further analysis may be performed on the amplification product to identify, quantify and analyze the detected virus (i.e., to determine parameters such as but not limited to the viral subtype/serovar, titre, pathogenecity, quantity etc.).

As used herein “preparing” or “preparing a sample” or “processing” or processing a sample” refers to one or more of the following steps to achieve separation of virus from sample components and in some embodiments optionally extraction and/or separation of a nucleic acid from a sample: (1) optional separation of viral cells from sample components, (2) optional viral enrichment, (3) optional viral lysis, and/or (4) optionally nucleic acid extraction and/or purification (e.g., DNA extraction, total nucleic acid extraction (i.e., DNA and RNA), genomic DNA extraction, RNA extraction). Types of nucleic acid extracted include, but are not limited to, DNA, RNA, mRNA and miRNA.

In some embodiments, a sample may not be processed but a viral nucleic acid can be detected in form of an viral RNA that is naturally present in the sample of an infected animal. For example, a reverse-transcriptase reaction can be performed on sample derived viral RNA followed by PCR amplification to amplify viral nucleic acids that are present in the sample.

As used herein, “presence” refers to the existence (and therefore to the detection) of a reaction, a product of a method or a process (including but not limited to, an amplification product resulting from an amplification reaction), or to the “presence” and “detection” of an organism such as a virus, viral subtype and/or a viral serovar.

As used herein, “detecting” or “detection” refers to the disclosure or revelation of the presence or absence in a sample of a target polynucleotide sequence or amplified target polynucleotide sequence product. The detecting can be by end point, real-time, enzymatic, and by resolving the amplification product on a gel and determining whether the expected amplification product is present, or other methods known to one of skill in the art.

The presence or absence of an amplified product can be determined or its amount measured. Detecting an amplified product can be conducted by standard methods well known in the art and used routinely. The detecting may occur, for instance, after multiple amplification cycles have been run (typically referred to an end-point analysis), or during each amplification cycle (typically referred to as real-time). Detecting an amplification product after multiple amplification cycles have been run is easily accomplished by, for instance, resolving the amplification product on a gel and determining whether the expected amplification product is present. In order to facilitate real-time detection or quantification of the amplification products, one or more of the primers and/or probes used in the amplification reaction can be labeled, and various formats are available for generating a detectable signal that indicates an amplification product is present. For example, a convenient label is typically a label that is fluorescent, which may be used in various formats including, but are not limited to, the use of donor fluorophore labels, acceptor fluorophore labels, flourophores, quenchers, and combinations thereof. Assays using these various formats may include the use of one or more primers that are labeled (for instance, scorpions primers, amplifluor primers), one or more probes that are labeled (for instance, adjacent probes, TaqMan® probes, light-up probes, molecular beacons), or a combination thereof. The skilled person in view of the present teachings will understand that in addition to these known formats, new types of formats are routinely disclosed. The present disclosure is not limited by the type of method or the types of probes and/or primers used to detect an amplified product. Using appropriate labels (for example, different fluorophores) it is possible to combine (multiplex) the results of several different primer pairs (and, optionally, probes if they are present) in a single reaction. As an alternative to detection using a labeled primer and/or probe, an amplification product can be detected using a polynucleotide binding dye such as a fluorescent DNA binding dye. Examples include, for instance, SYBR® Green dye or SYBR® Gold dye (Molecular Probes). Upon interaction with the double-stranded amplification product, such polynucleotide binding dyes emit a fluorescence signal after excitation with light at a suitable wavelength. A polynucleotide binding dye such as a polynucleotide intercalating dye also can be used.

The present disclosure relates in general to methods and kits for detecting and diagnosing BVDV infected animals. BVDV is an approximately 12 kb single-stranded, positive RNA enveloped virus in the genus Pestivirus of the Flaviviridae family. BVDV infection in cattle is a worldwide problem that results in significant economic losses in the dairy and beef industries. It can cause respiratory disease, enteritis, still-birth, abortion, and mucosal disease. In utero BVDV infection can induce immunotolerance, causing animals to be persistently infected (PI) for life. PI animals continuously shed the virus and are the main source of BVDV infection in herds. Rapid detection of PI cattle is essential for BVDV control.

All currently existing methods for diagnosing/determination of PI animals requires two positive results from the same animal taken at least 3 weeks apart for distinction between PI and TI animals. Methods of BVDV detection may involve one or more methods such as Antigen Capture ELISA (ACE), Immunohistochemistry (IHC), Virus Isolation (VI) and/or qRT-PCR. However, irrespective of the method of BVDV testing, methods for determining PI BVDV infection require two tests at least 3 weeks apart as TI animals clear the BVDV from their bodies in 3 weeks.

The present disclosure, in some embodiments, describes methods for diagnosing/determining a PI animal comprising a one-time BVDV test. Methods of the disclosure therefore provide better, faster and more economical methods for discriminating between PI and TI animals. Methods for diagnosing/determining a PI animal described herein avoid the need to test an animal for a second time after 3 weeks to confirm PI. This greatly reduces the logistical and economic burden by eliminating the need to re-test animals.

In some embodiments, a method of the disclosure for diagnosing an animal as persistently infected (PI) or transiently infected (TI) with a bovine viral diarrhea virus (BVDV) comprises: a) performing a single BVDV-specific polymerase chain reaction (PCR) test comprising: 1) obtaining a sample from an animal suspected of being infected with BVDB; 2) contacting nucleic acids in the sample with at least one primer pair specific to hybridize to and amplify a BVDV-specific target nucleic acid; 3) amplifying at least one BVDV-specific nucleic acid from the sample nucleic acids by providing appropriate amplification conditions; 4) detecting one or more BVDV-specific amplification products; and b) determining Ct value of the BVDV-specific amplification product, wherein a Ct value of ≦31 indicates that the animal is a PI animal and a Ct value of >31 indicates that the animal is a TI animal. In some embodiments of the method described here, a Ct value in the range of from about 32 to about 40 is indicative of TI in the tested animal, and a Ct value in the range of from about 20 to about 31 is indicative of PI in the tested animal.

The Ct value which corresponds to the BVDV titer has been found by the present inventors to be different in PT and TI animals. Viral titer in a skin biopsy or in ear notch samples has been presently found to be high in a PI animal but low in a TI animal, even at the peak of infection.

The present methods are based on determination, by the present inventors, of a suitable diagnostic cut-off Ct value and/or value range for presumptive PI cattle obtained from cattle derived samples from a mixed population of cattle including uninfected, persistently infected (PI) and transiently/acutely infected (TI) animals.

In some embodiments, a method of the disclosure can comprise a reverse-transcriptase-polymerase chain reaction (RT-PCR) to detect BVDV specific RNA present in a sample. BVDV-specific RNA can be reverse transcribed to a cDNA and then amplified by PCR for detection of BVDV-specific target nucleic acids.

In one embodiment, the disclosure also describes methods to determine the type of a BVDV infection in an animal, i.e., a PI or a TI BVDV infection. Such a method comprises performing either an RT-PCR based detection of BVDV RNA in a sample obtained from an animal by forming a BVDV-specific amplified product; or performing an PCR based detection of BVDV-specific nucleic acids (DNA) in a sample obtained from an animal; and determining/calculating the Ct value of one or more BVDV-specific amplification product formed by the PCR, wherein a Ct value of ≦31 is indicative of a PI animal and a Ct value of >31 is indicative of a TI animal.

In some embodiments, a method to determine the type of a BVDV infection in an animal, i.e., a PI or a TI BVDV infection comprises performing either an RT-PCR based detection of BVDV RNA in a sample obtained from an animal as described above; or performing a PCR based detection of BVDV nucleic acids (DNA) in a sample; and determining/calculating the Ct value of the BVDV titer wherein a Ct value in the range of from about 32 to about 40 is indicative of TI in the tested animal, and a Ct value in the range of from about 20 to about 31 is indicative of PI in the tested animal.

A method of the disclosure, in one embodiment, is a method to detect a PI animal and comprises: 1) optionally performing an RT reaction on sample derived nucleic acids to convert sample RNA into cDNA; 2) contacting nucleic acids (DNA/cDNA/genomic DNA) from a sample derived from an animal with at least one primer set that is specific to hybridize to and amplify BVDV-specific target nucleic acids; 3) performing an PCR on nucleic acids from the sample hybridized to the at least one primer set; 4) detecting one or more BVDV-specific amplification products; and 5) determining Ct value of the BVDV-specific amplification product, wherein a Ct value of ≦31 indicates that the animal is a PI animal. In some embodiments a Ct value in the range of from about 20 about 31 indicates an animal is PI with BVDV.

In one embodiment, a method for determining a PI animal comprises RT-PCR based detection of BVDV RNA as described above and calculating the Ct value of the BVDV amplification product wherein a Ct value in the range of from about 20 to about 31 is indicative of PI in the tested animal.

A method of the disclosure, in one embodiment, is a method to detect a TI animal and comprises: 1) optionally performing an RT reaction on sample derived nucleic acids to convert sample RNA into cDNA; 2) contacting nucleic acids (DNA/cDNA/genomic DNA) from a sample derived from an animal with at least one primer set that is specific to hybridize to and amplify BVDV-specific nucleic acids; 3) performing an PCR or an RT-PCR on nucleic acids from the sample hybridized to the at least one primer set; 4) detecting one or more BVDV-specific amplification products; and 5) determining Ct value of the BVDV-specific amplification product, wherein a Ct value of >31 indicates that the animal is a TI animal. In some embodiments, a Ct value in the range of from about 32 to about 40 indicates an animal is a TI infected with BVDV. In one embodiment, the disclosure describes methods to diagnose/determine a TI animal.

In one embodiment, a method for diagnosing/determining a TI animal comprises RT-PCR based detection of BVDV RNA comprising forming a BVDV specific amplification product and calculating the Ct value of the BVDV-specific amplification product, wherein a Ct value of >31 is indicative of a TI animal.

In one embodiment, a method for diagnosing/determining a TI animal comprises RT-PCR based detection of BVDV RNA as described above and determining the Ct value of the BVDV-specific amplification product, wherein a Ct value in the range of from about >31 to about 40 (if 40 PCR cycles are run) is indicative of TI in the tested animal.

In some embodiments, a method to distinguish if a BVDV infection in an animal is a PI or a TI BVDV infection according to the present disclosure comprises: 1) contacting nucleic acids from a sample derived from an animal with at least one primer set that is specific to hybridize to and amplify BVDV-specific nucleic acids; 2) performing an PCR (and optionally an RT prior to the PCR) on nucleic acids from the sample hybridized to the at least one primer set; 3) detecting one or more BVDV-specific amplification products; and 4) determining Ct value of the BVDV-specific amplification product, wherein a Ct value of >31 indicates that the animal is a TI animal and a Ct value ≦31 indicates that the animal is a PI animal.

In some embodiments, a method to distinguish if a BVDV infection in an animal is a PI or a TI BVDV infection according to the present disclosure comprises: 1) contacting nucleic acids from a sample derived from an animal with at least one primer set that is specific to hybridize to and amplify BVDV-specific nucleic acids; 2) performing an PCR (or an RT prior to the PCR and the “1) contacting step”) on nucleic acids from the sample hybridized to the at least one primer set; 3) detecting one or more BVDV-specific amplification products; and 4) determining Ct value of the BVDV-specific amplification product, wherein a Ct value in the range of from about 32 to about 40 is indicative of TI in the tested animal, and a Ct value in the range of from about 20 to about 31 is indicative of PI in the tested animal.

Animals that can be tested and/or diagnosed by a method include any mammal that can be afflicted with a BVDV. Non limiting examples include animals such as cattle, sheep, goats, deer and/or alpacas.

A sample derived from an animal to be tested for BVDB can be an ear notch sample, a hair sample, a tail hair sample, a serum sample, a blood sample, a milk sample, a urine sample, a fecal sample, a skin sample, a lymph sample, a plasma sample, a cerebrospinal fluid sample, a mucus sample, a throat swab sample, and/or any sample from a bodily fluid or tissue from an animal suspected of having BVDV. In some embodiments, ear notch, skin, hair and milk samples are preferable.

Methods of the disclosure are applicable for diagnosing PI and TI animals regardless of the BVDV sub-type afflicting the animal. For example, methods described here can be used to detect various BVDV sub-types for PI or TI infection such as but not limited to a BVDV subtype 1A, a BVDV subtype 1B and/or a BVDV subtype 2A.

In some embodiments, determining a Ct value comprises calculating the BVDV titer in a sample.

In some embodiments, kits for detecting BVDV, according to the disclosure may provide instructions to test for Ct values as described or Ct ranges as described to make a diagnosis of TI or PI in BVDV animals. In some embodiments, kits of the disclosure can have software components that can calculate the Ct value or Ct range and output the results as PI or TI positive (or negative) on a screen or in a printout. Software programs can be part of RT-PCR or PCR machines and can comprise a GUI for user friendly steps for calculations and analysis.

In some embodiments, the methods described above can comprise: 1) obtaining nucleic acids from a cattle derived sample obtained from an animal that is to be tested for type of BVDV infection, i.e. PI infection or TI infection (such as but not limited to ear notch samples, hair samples, tail hair samples, blood samples, urine samples, fecal samples, skin samples, lymph samples, plasma samples, serum samples, cerebrospinal fluid samples, mucus samples, throat swabs, milk samples and/or any bodily fluid or tissue); 2) performing an RT-PCR (or other nucleic acid amplification reaction) on the nucleic acids derived from the sample to detect viral nucleic acids (such as BVDV RNA, BVDV DNA, BVDV cDNA, BVDV genomic DNA); 3) if the sample is detected to be positive for BVDV (has viral RNA/DNA or other viral nucleic acids), then performing calculation of viral (BVDV) titer to determine if an animal is PI or TI. Calculation of BVDV titer may be performed by calculating the Ct values of the PCR reaction of a known standard curve. Accordingly, in some embodiments, a qPCR method may be used. In some embodiments, an ear notch sample may be preferred.

In some embodiments, a method of the disclosure can comprise using an existing BVDV detection kit such as Life Technologies' VetMAX™-Gold BVDV Detection. This kit is be used for rapid detection of BVDV RNA purified from bovine samples (such as but not limited to bovine ear notch samples) and is optimized for real-time RT-PCR amplification of both Xeno™ RNA internal positive control and BVDV RNA targets. Kit components normally comprise an RT-PCR buffer, RT-PCR enzyme mix, BVDV specific primers and probes, Xeno™ RNA internal positive control and other agents such as solutions to dilute nucleic acids and nuclease-free water.

In one embodiment, a method according to the disclosure can further comprise processing a sample obtained from an animal suspected of being infected with a BVDV to obtain sample derived nucleic acids (such as RNA/DNA) and can comprise treating the sample with a composition operable to lyse the sample and the virus in the sample thereby creating a lysate comprising viral nucleic acids; separating viral nucleic acids from other components of the lysate.

In some embodiments, nucleic acids isolated from a sample may include those from a BVDV (if present) and the bovine/animal (from which sample was obtained) nucleic acids as well as from other living organisms that may be associated with (present in) a sample being analyzed. For example, some bovine samples may comprise microbes of a normal flora, a commensal organism or a mutualistic flora in addition to bovine nucleic acids.

Methods of the disclosure may be amenable to isolate any nucleic acids including but not limited to DNA, RNA (mRNA, rRNA, tRNA, miRNA, piRNA, mitochondrial RNA), or any type of nucleic acid.

Accordingly methods may comprise optional steps such as mixing, stifling and/or agitating a lysis buffer into a sample to ensure contact of lysis buffer with all components of the sample. This step may also comprise incubating a sample and a lysis buffer under conditions to lyse cells and form a lysate. Conditions may comprise incubating at certain temperatures and/or for certain time lengths. A method may also comprise separating viral nucleic acids from other components of the sample and may include one or more of the following non-limiting steps such as washing and/or centrifugation and/or precipitation and/or binding to columns and/or elution any combinations thereof.

These steps may be modified slightly, using one or more methods to isolate nucleic acids known to those skilled in the art, in view of this disclosure. Accordingly, slight variations using methods known in the art, in light of this disclosure are within the scope of this disclosure. See, for example, Chomczynski and Sacchi (1987) for methods of isolating RNA. Methods of the present disclosure, if isolating RNA, may employ the use of a Trizol reagent (Gibco Life Technologies) to extract total RNA. The Trizol procedure involves homogenization of a sample in a blender followed by extraction with a phenol-based Trizol reagent. RNA is then precipitated with isopropyl alcohol and washed with ethanol before being redissolved in RNAse-free water or 0.5% SDS.

Other methods that may be employed for RNA isolation may use of products known in the art such as RNAzol (Gibco BRL), TriReagent™ (Molecular Science), Qiagen's RNeasy™ Total RNA Isolation kit (Qiagen), Quickprep™ Total RNA Extraction kit (Amersham Bioscience) or any other manufactured protocol for isolation of RNA. Other methods of RNA extraction, may include but are not limited to, the guanidine thiocyanate and cesium trifluoroacetate (CSTFA) method, the guanidinium hydrochloride method, or the lithium chloride-SDS-urea method. See Sambrook et al. (2000); Maniatis et al. (1989); Ausubel et al. (1994), for example of methods of RNA extraction.

The methods may be performed manually, automatically and/or by a combination of manual and automatic steps.

In some embodiments, calculation of viral titer and determination of Ct values may be performed by a computer system using a software. A computer system may comprise a central processing unit, hardware and software elements operable to control and direct any automated steps of sample processing (by automated devices that may be used for one or more steps such as sample processing and/or detection of BVDV nucleic acids and/or calculation of Ct values. In some embodiments, a computer system may include pre-loaded software and/or Application Specific Integrated Circuits (ASICS) that may enable the sample processing and analysis steps of the methods of the disclosure and may also control displaying and/or exporting the results. In some embodiments, a user will be able to easily review the end result which may simply indicate that the sample tested is from an animal that is a PI animal or a TI animal. Computer and software systems of the disclosure may enable a user with no knowledge of PCR to very simply know the results of a test.

In some embodiments, Life Technologies' VetMAX™-Gold BVDV Detection Kit was used for rapid detection of BVDV RNA purified from bovine samples (such as but not limited to bovine ear notch samples) obtained from 71 crossbred animals between 6-12 months of age from feedlots in Kansas screened to be positive by ACE. Of these 63 animals were confirmed to be PI with positive tests by at least two confirmatory methods (ACE, IHC, VI) three weeks after initial positive ACE test (i.e., by traditional PI testing methods that using testing twice, second test after 3 week) and further tested by qRT-PCR. Of these, 10 PI animals selected from this population were used in a commingling study (also described later in the section entitled Examples).

53 BVDV negative animals were commingled with 10 PI animals over a period of 20 days to make TI animals. Serum was collected throughout study and ear punches collected at days −2, 8, 13, and 20 due to animal welfare concerns.

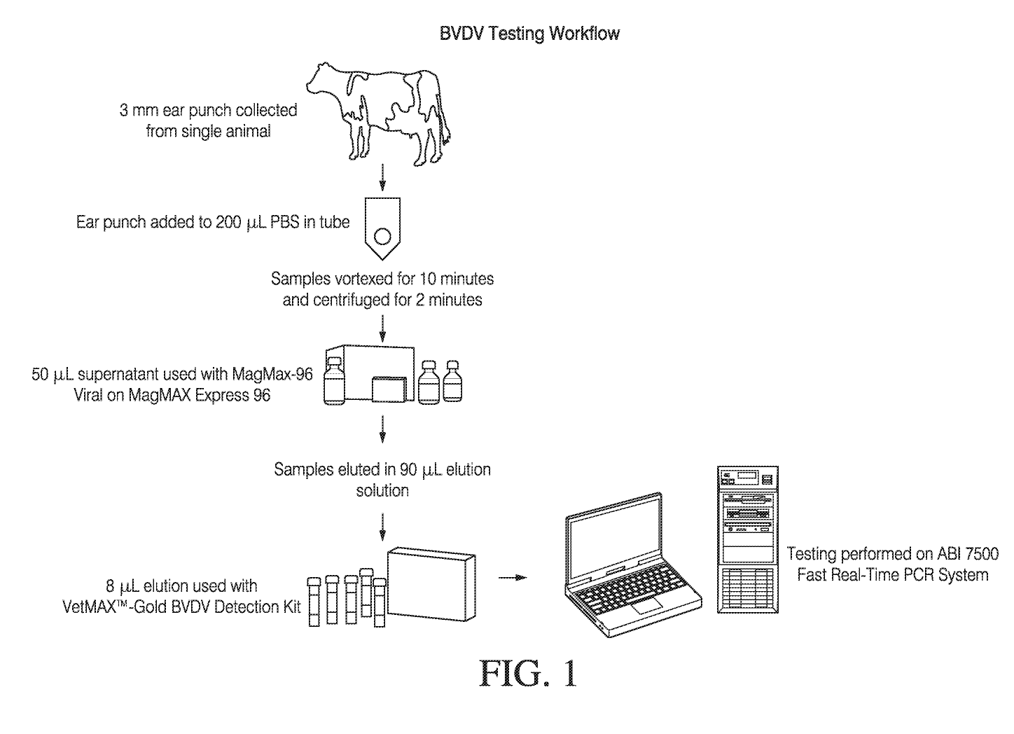

A BVDV testing workflow is described in FIG. 1 using MagMax-96 Viral on MagMAX Express 96 kits and a VetMAX™-Gold BVDV Detection Kit from Life Technologies and testing was performed on an ABI 7500 Fact Real-Time PCR system.

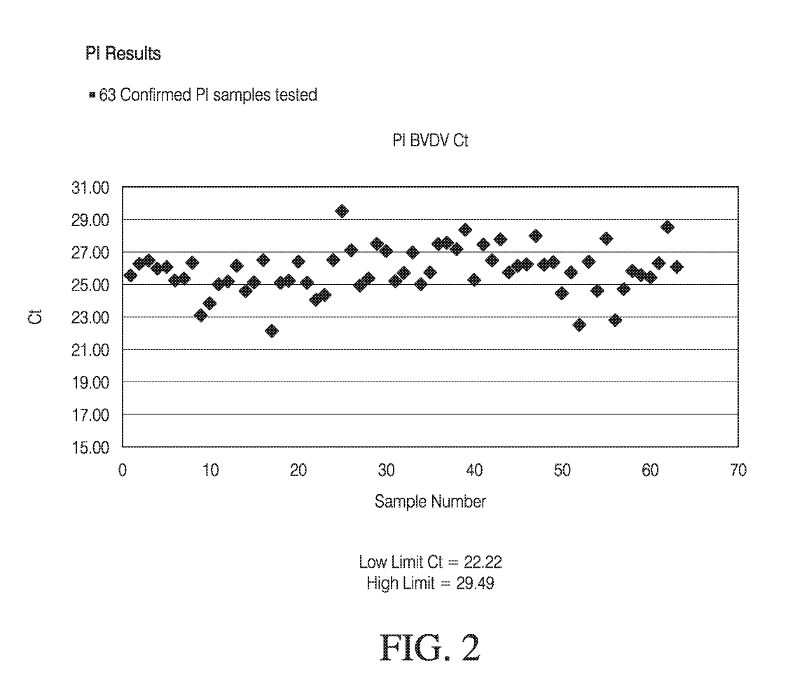

FIG. 2 shows the Ct values of the 63 confirmed PI samples that were tested, showing a low limit of Ct as 22.22 and a high limit of Ct as 29.49.

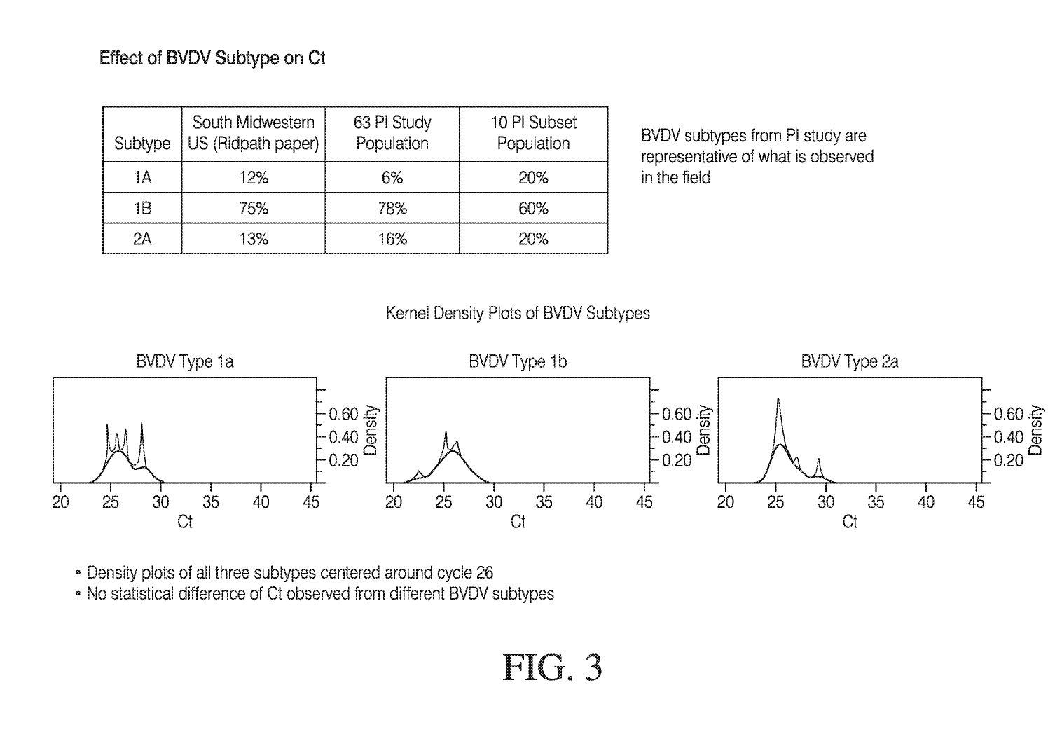

Ct values of PI animals were also determined for different BVDV subtypes. For example, BVDV subtypes 1A, 1B and 2A were tested for Ct values in animals that were confirmed by traditional methods (using testing twice, second test after 3 weeks) to be PI animals. These results are shown in FIG. 3, which shows that no statistical differences are see in Ct values from different BVDV subtypes.

In some embodiments, the disclosure also provides methods to determine if an animal has a PI or a TI-type BVDV infection, irrespective of the type of BVDV subtype that an animal may be infected with. For example, such a method may comprise performing an RT-PCR based detection of a BVDV nucleic acids (e.g., RNA) in a sample obtained from an animal; and calculating the Ct value of the BVDV titer wherein a Ct value in the range of from about 20 to about 31 is indicative of PI in the tested animal, and a Ct value in the range of from about 32 to about 40 is indicative of TI in the tested animal. In some embodiments, a PI animal may be determined by Ct values centered around cycle 26 (see FIG. 3). In some embodiments of such a method a BVDV infecting the animal may be BVDV subtype 1A, BVDV subtype 1B and BVDV subtype 2A.

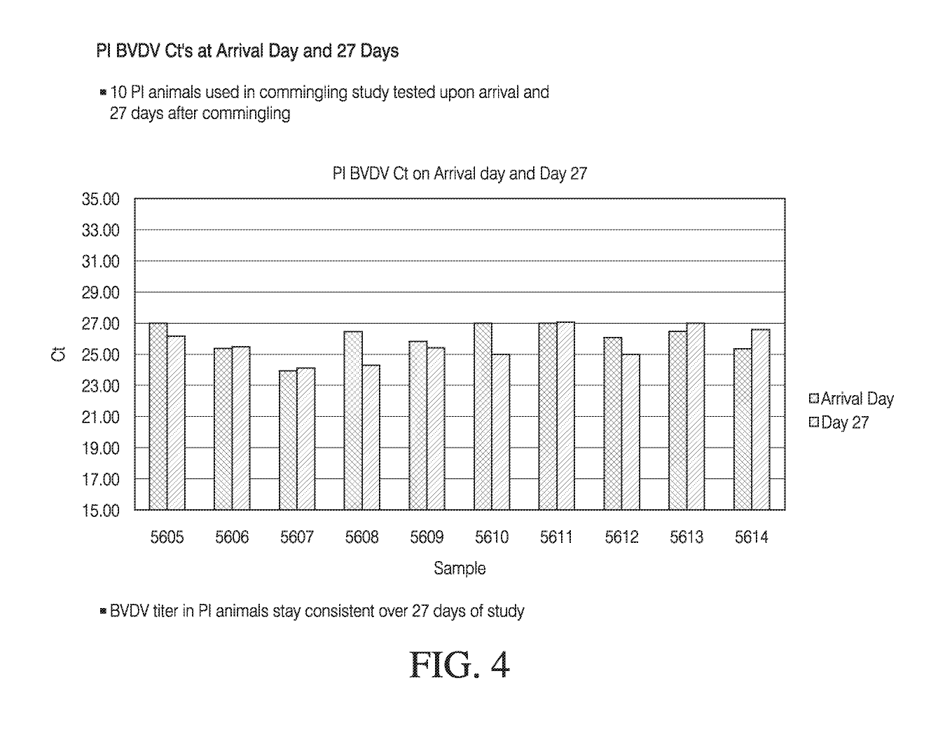

Additionally, in some embodiments of a method of the disclosure the Ct value or Ct value range described above may be monitored at any day. For example, FIG. 4 depicts results of determining Ct values in PI animals found to be PI by traditional methods on the arrival day (first day animals were obtained) and on the 27^{th} day. 10 PI animals were tested on arrival day and at day 27 and BVDV titers were found to stay consistent in the animals over the 27 days (see FIG. 4).

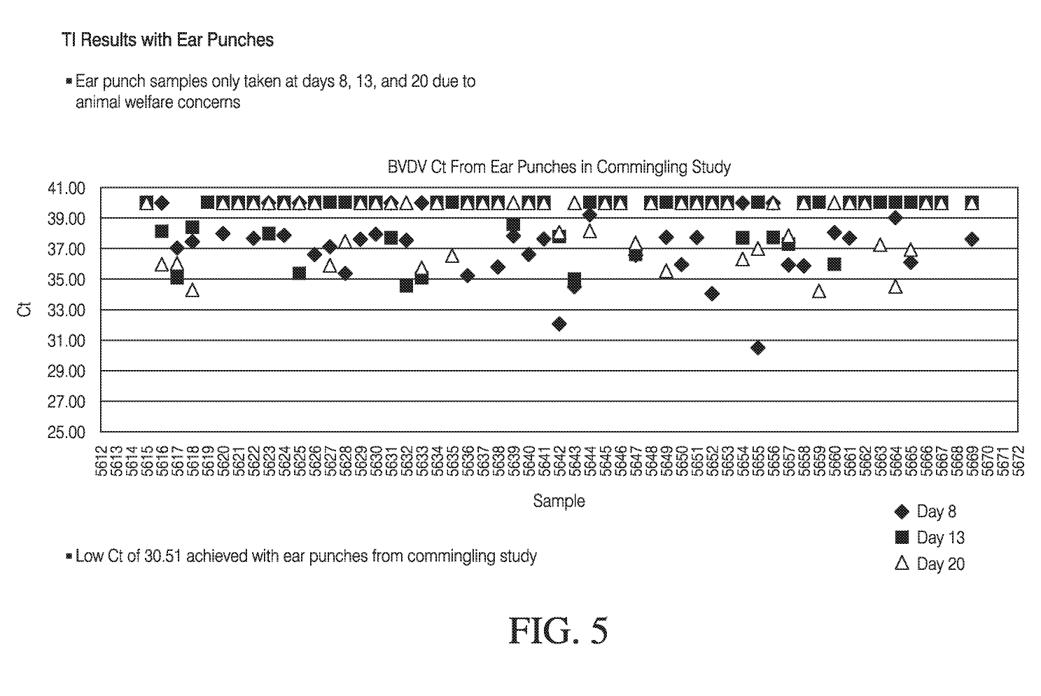

In the comingling study where 53 BVDV negative animals were commingled with 10 PI animals over a period of 20 days to make TI animals. Ear punches collected at days −2, 8, 13, and 20 of the TI animals were analyzed for Ct values. FIG. 5 shows the Ct values to be in no lower than 30.51 for the TI animals.

In some embodiments, the disclosure also provides methods to determine if an animal has a PI or a TI-type BVDV infection, comprising performing an RT-PCR based detection of a BVDV nucleic acids (e.g., RNA) in a sample obtained from an animal; and calculating the Ct value of the BVDV titer wherein a Ct value no lower than 30.51 is indicative of a TI animal.

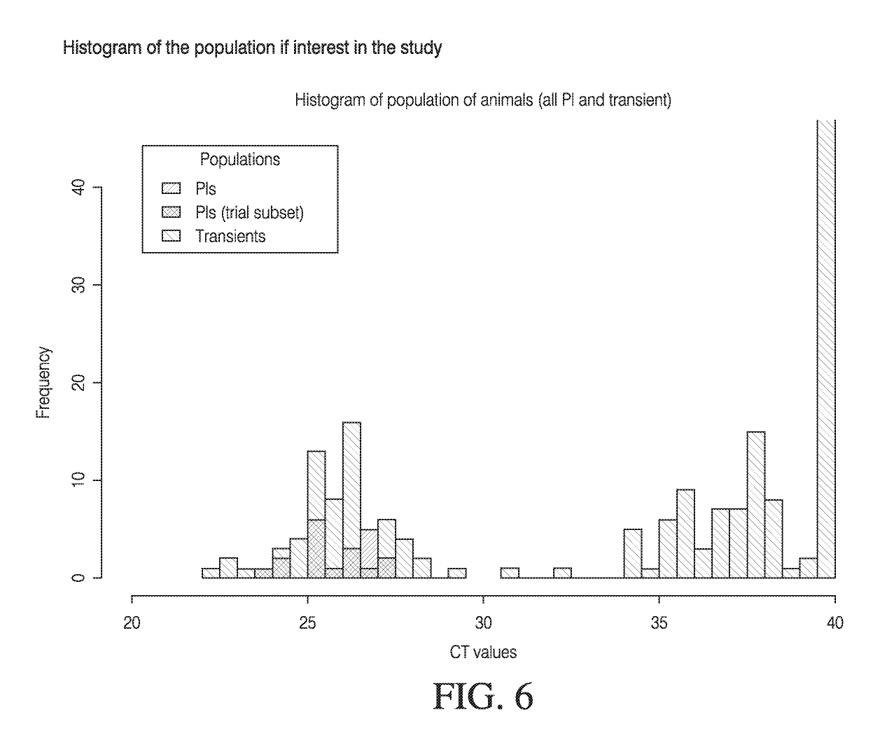

Example 1 and FIG. 6 show that there is no overlap of PI and TI Ct values between the ranges of from about 29.49 to about 30.51.

The section entitled Examples describes several examples of methods and workflow protocols tested using methods, and/or apparatus, and/or systems and/or compositions of the disclosure.

The present disclosure also describes kits and/or software for implementing the methods discussed herein. A kit of the disclosure may also comprise one or more reagents and buffers for extracting, isolating and/or purification of nucleic acids from a sample and a instructions/software for calculating Ct values.

A kit may further comprise reagents for downstream processing of an isolated nucleic acid and may include without limitation at least one RNase inhibitor; at least one cDNA construction reagents (such as reverse transcriptase); one or more reagents for amplification of RNA, one or more reagents for amplification of DNA including primers, reagents for purification of DNA, probes for detection of specific nucleic acids. A kit of the disclosure may in some embodiments include components for the identification of a BVDV in a sample comprising primer/probes having sequences specific to a BVDV. Various subtypes and serovars of BVDV may be detected.

Reagents and components of kits may be comprised in one or more suitable container means. A container means may generally comprise at least one vial, test tube, flask, bottle, syringe or other container means, into which a component may be placed, and preferably, suitably aliquoted. Where there are more than one component in a kit they may be packaged together if suitable or the kit will generally contain a second, third or other additional container into which the additional components may be separately placed. However, in some embodiments, certain combinations of components may be packaged together comprised in one container means. A kit can also include a means for containing the DNA/RNA, and any other reagent containers in close confinement for commercial sale. Such containers may include injection or blow-molded plastic containers into which the desired vials are retained.

A kit of the disclosure may also include instructions for employing the kit components and may also have instructions for the use of any other reagent not included in the kit. Instructions can include variations that can be implemented.

### EXAMPLES

Aspects of the present teachings can be further understood in light of the following examples, which should not be construed as limiting the scope of the present teachings in any way.

### Example 1

### Establishment of Presumptive Diagnostic Cut-Off for Persistently Infected Cattle

The Bovine Virus Diarrhea RNA Test Kit is a simple, rapid, and highly sensitive one-step real-time reverse transcription PCR (RT-PCR) assay for the diagnosis of Bovine Viral Diarrhea (BVD) in cattle through the detection of BVDV RNA isolated from ear notch samples and is sold by Life Technologies as the VetMAX™-Gold BVDV Detection Kit.

The assay is a single-tube, one-step reaction in which RNA is reverse transcribed into cDNA and a BVDV target is amplified using AmpliTaq Gold® UP (Ultra Pure) DNA Polymerase. Amplification products are detected in real-time using fluorescent TaqMan® probes (hydrolysis probe chemistry). The assay can consistently detect as few as 40 copies of BVDV RNA. The reagents also include both an internal positive control (XenoRNA-01), and a BVDV Control RNA to monitor RT-PCR efficiency and inhibition.

In the present study, Life Technologies Bovine Virus Diarrhea RNA Test Kit (Applied Biosystems, PN 4413938) was evaluated to determine if a suitable diagnostic cut-off for presumptive PI cattle could be established for ear notch samples obtained from a mixed population of cattle including uninfected, persistently infected (PI) and transiently/acutely infected animals characterized by various methods.

After confirming that an assay cut-off could be established, statistical analysis of study data was performed to determine a suitable one-sided upper C_{T} cut-off for detection of presumptive PI cattle.

### Materials Used

The following materials and systems were used to test cattle samples as described in Table 1: 1) Bovine Virus Diarrhea RNA Test Kit (Applied Biosystems, P/N 4413938), USDA Product Code 5A06.80, Lot 0910003; 2) Purified RNA obtained from characterized BVDV acutely infected and PI cattle (sample information in Tables 1-2); 3) Applied Biosystems 7500-Fast Real-Time PCR System (96-Well) and PC with SDS v1.4 data analysis software; 4) MicroAmp Fast Optical 96-Well Reaction Plates, PN 4346906; 5) MicroAmp Optical 96-Well Reaction Plates, PN N801-0560; and 6) MicroAmp Optical Adhesive Film, PN 4311971.

TABLE 1
Cattle tested to Distinguish PI from Acutely Infected Cattle

| Cattle tested | Number Tested |
|---|---|
| PI | 71 |
| Confirmed BVDV Negative | 53 |
| Total Cattle (PI and Negative) | 124 |

TABLE 2
Sample Matrices Tested

| | | Number of Samples Tested |
|---|---|---|
| Confirmatory Method* | Sample Type | Positive for BVDV |
| Antigen Capture ELISA | Serum | 63 out of 71 |
| (ACE) | | |
| Immunohistochemistry (IHC) | Serum | 64 out of 71 |
| Virus Isolation (VI) | Buffy Coat | 61 out of 71 |

*For PI cattle, positive ACE samples from the field were re-tested with VI, IHC and a second ACE test. These samples were assigned PI status when calculating C_{T} cut-off for PI status.

A. Recruitment and Characterization of BVDV PI Animals and Selection of Subset for Commingling Study:

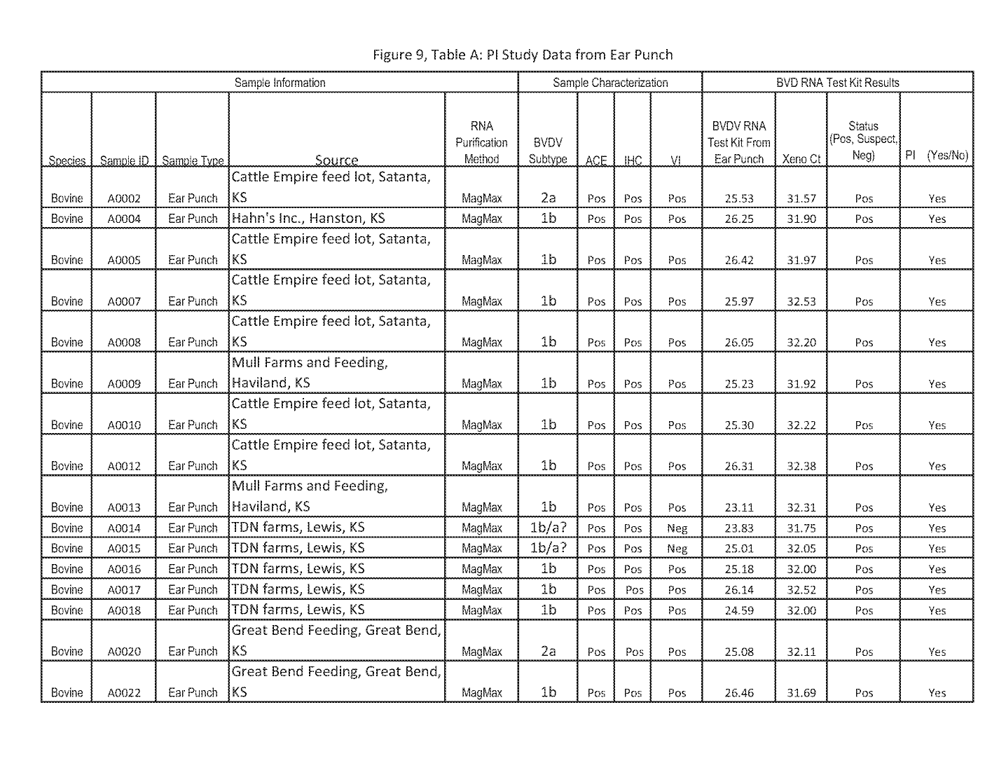

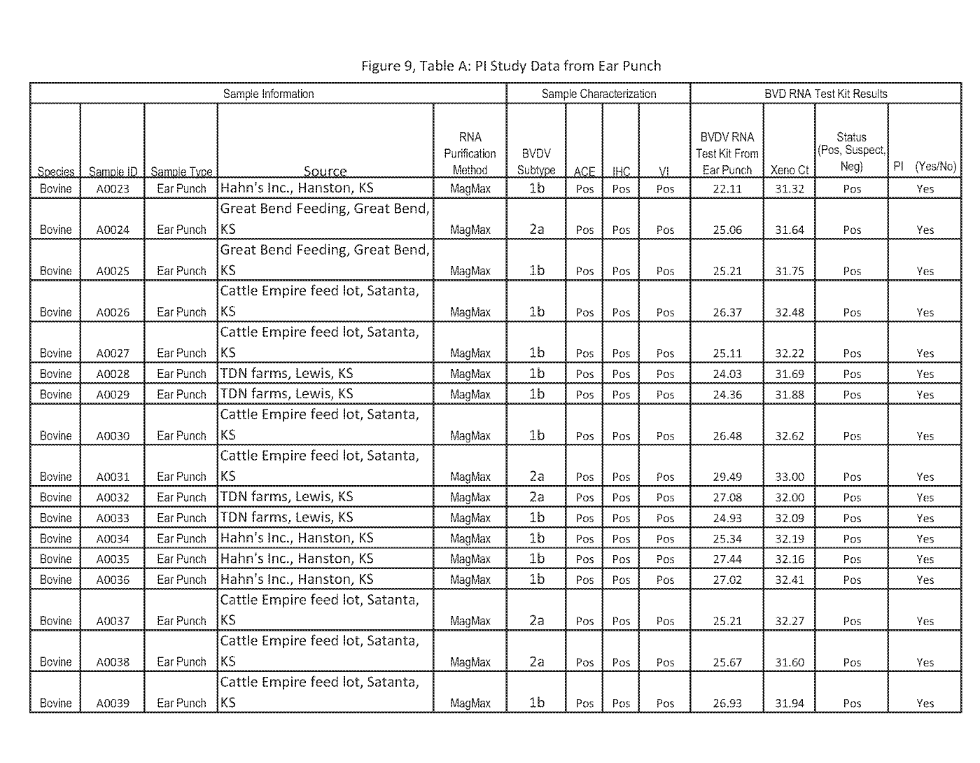

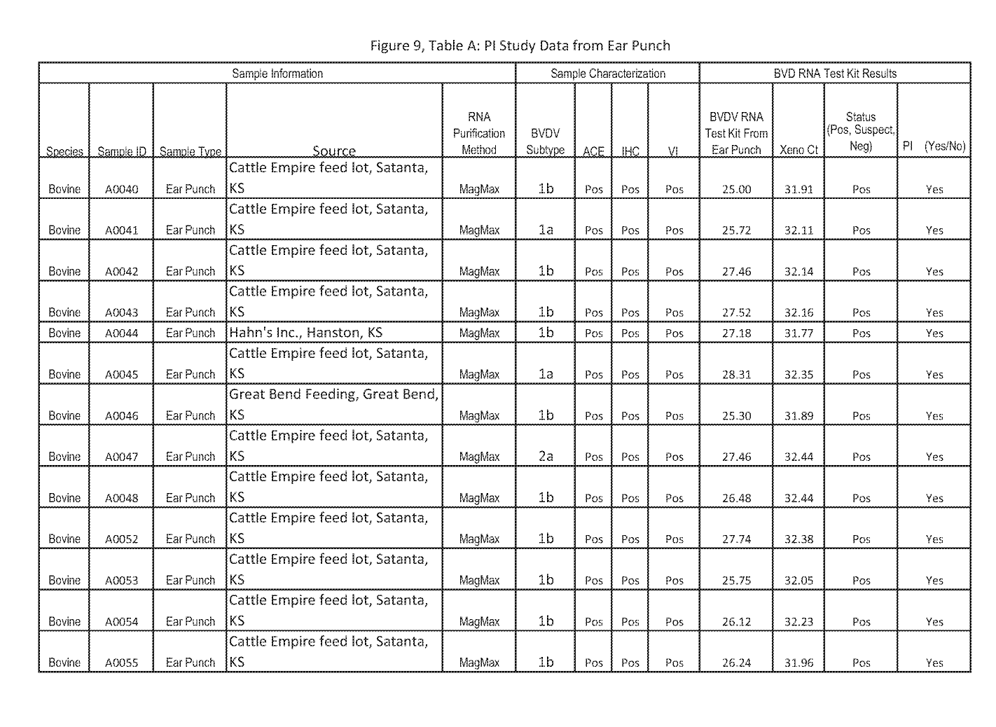

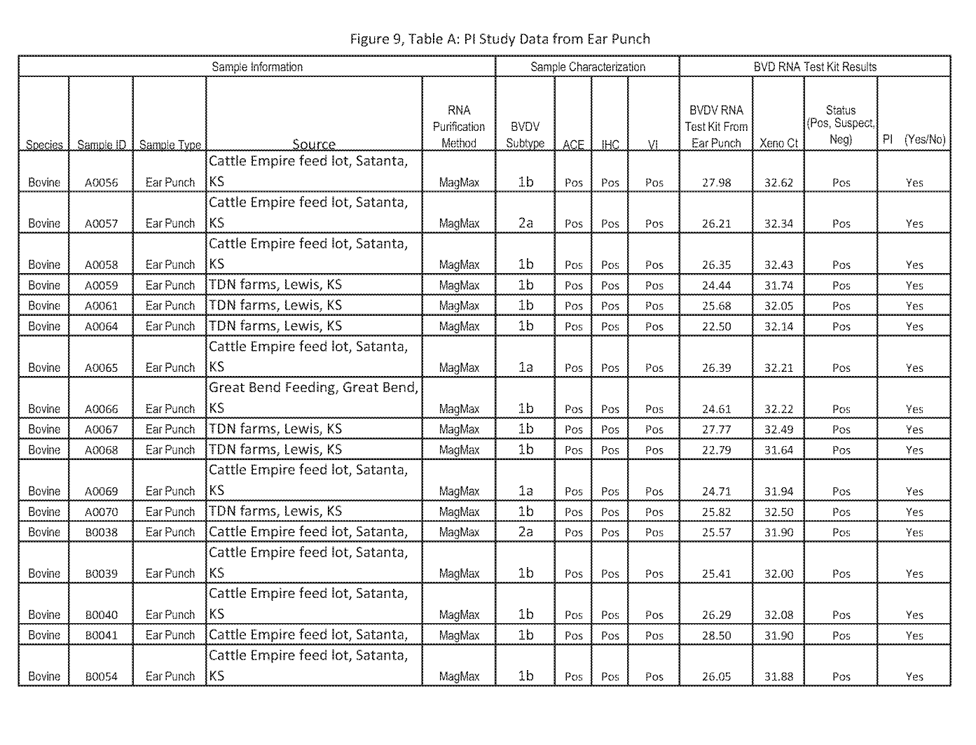

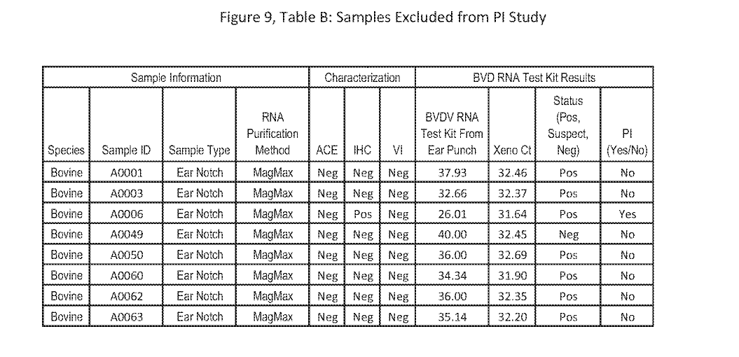

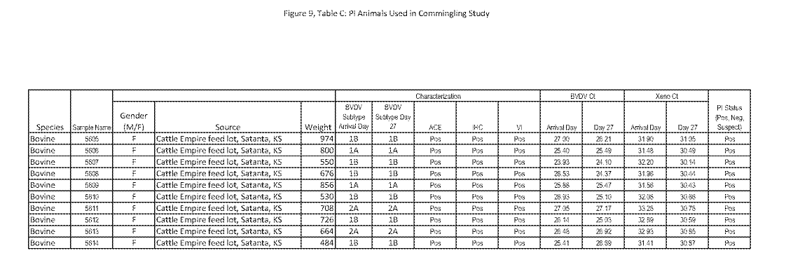

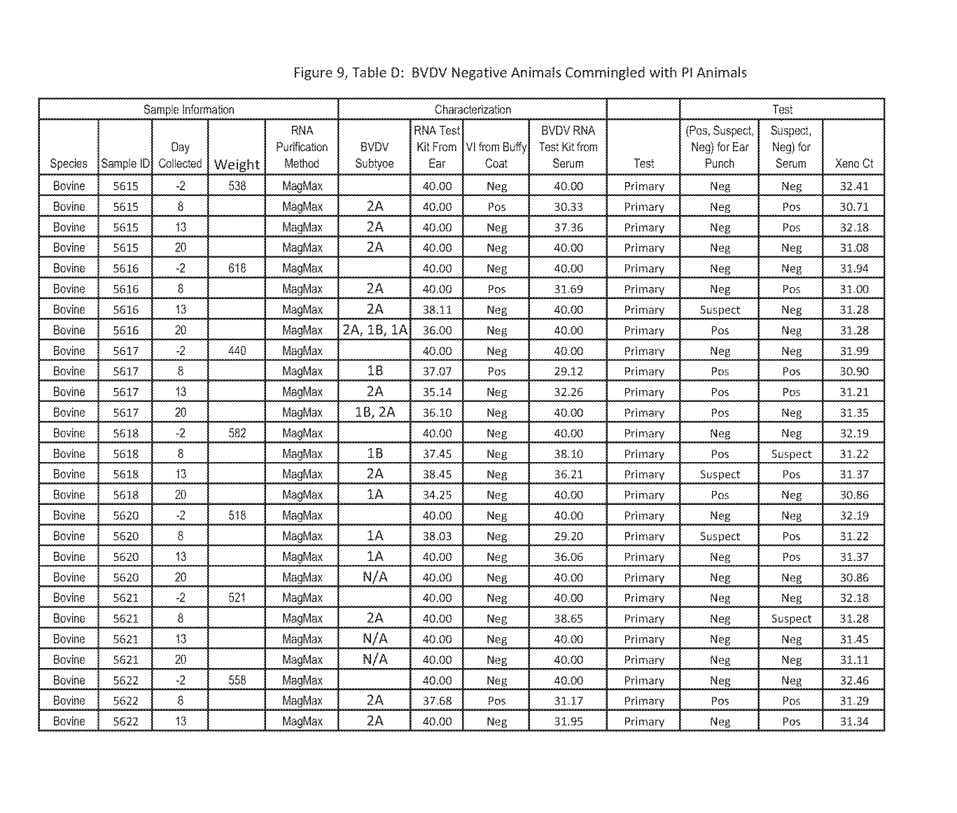

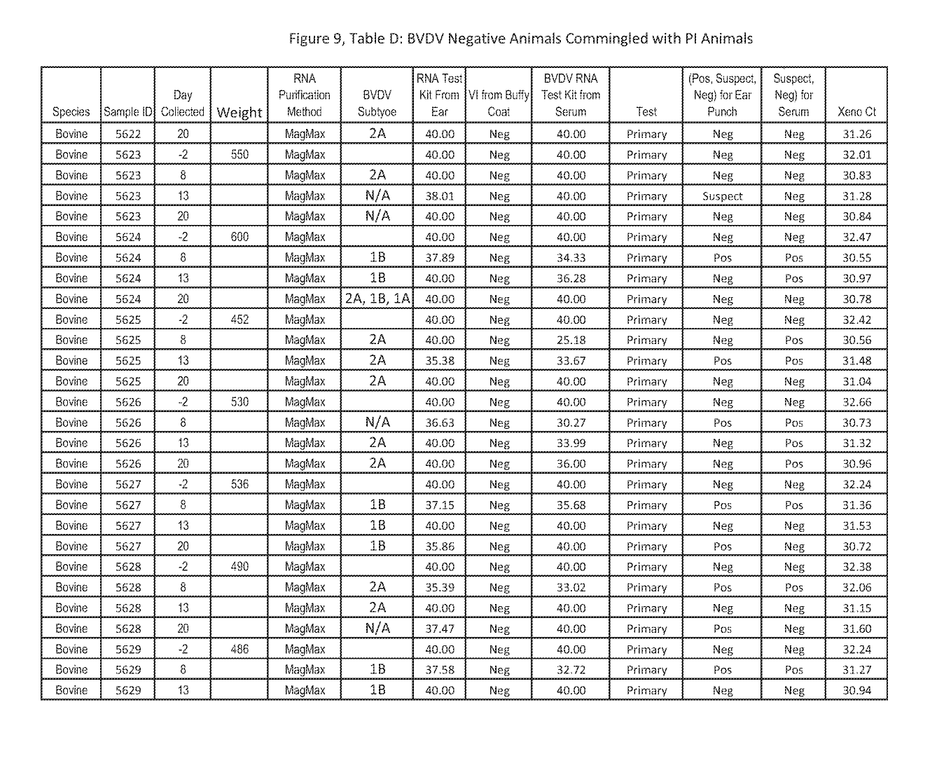

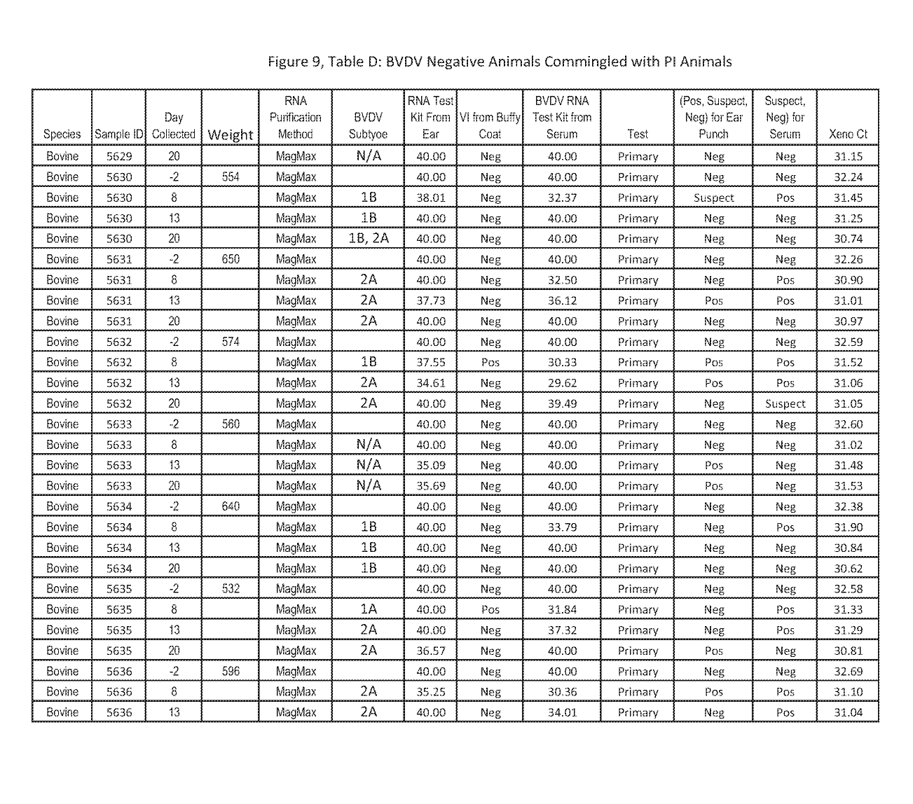

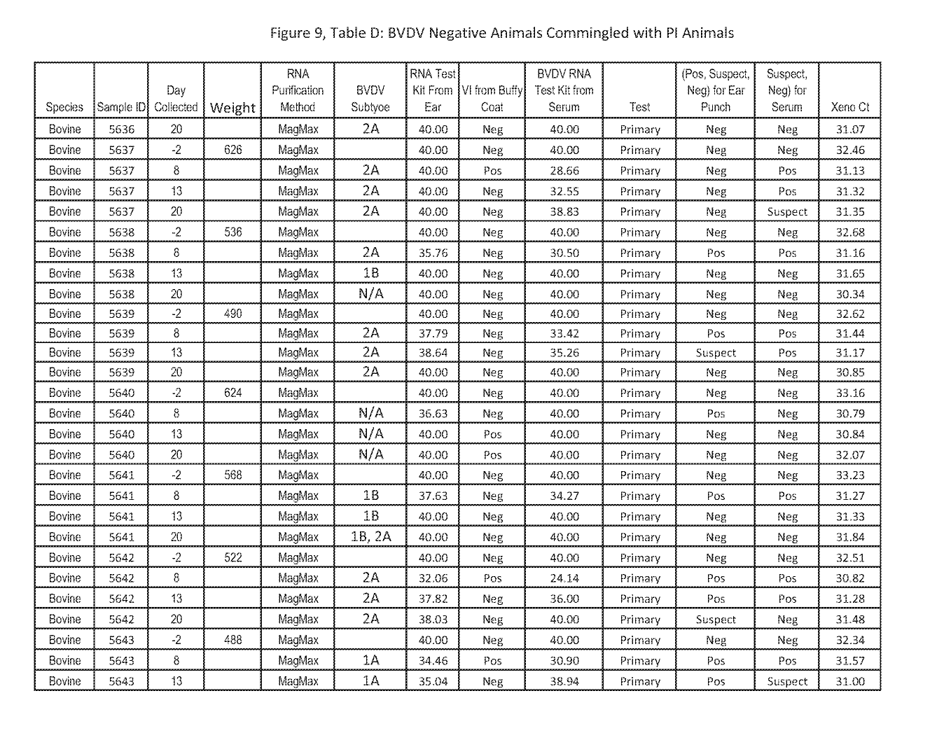

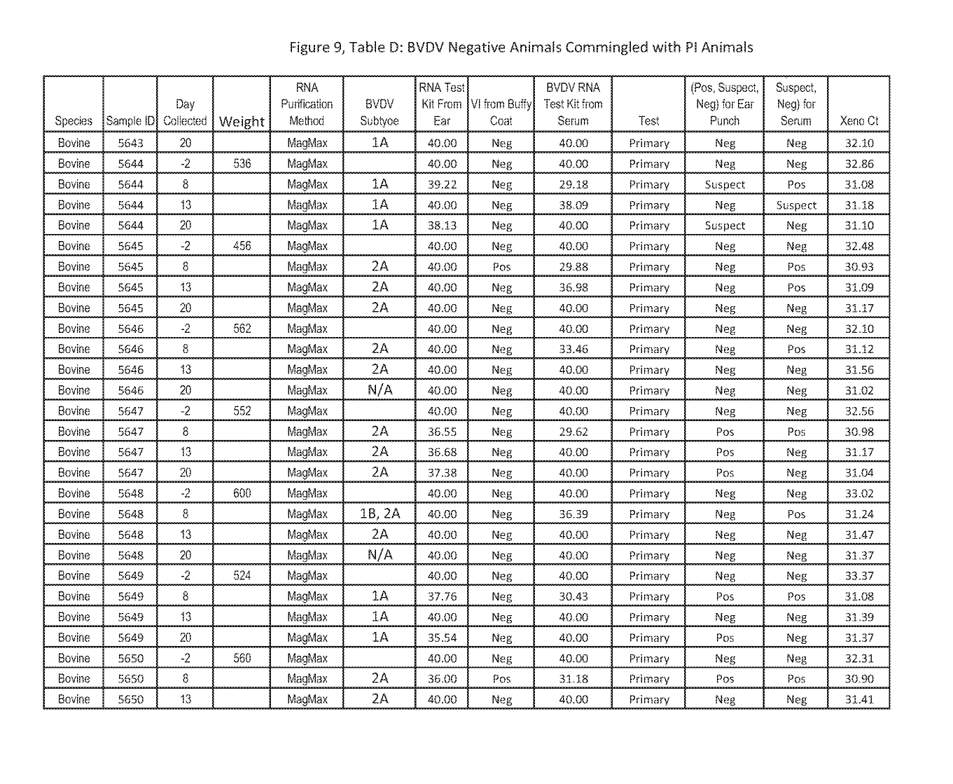

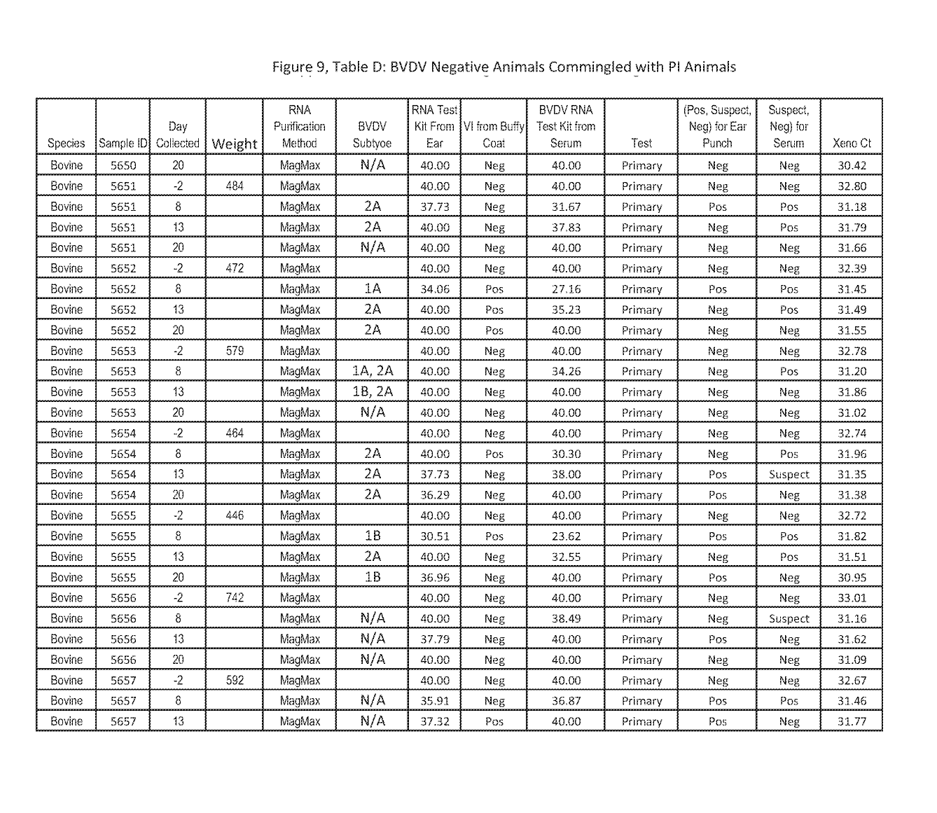

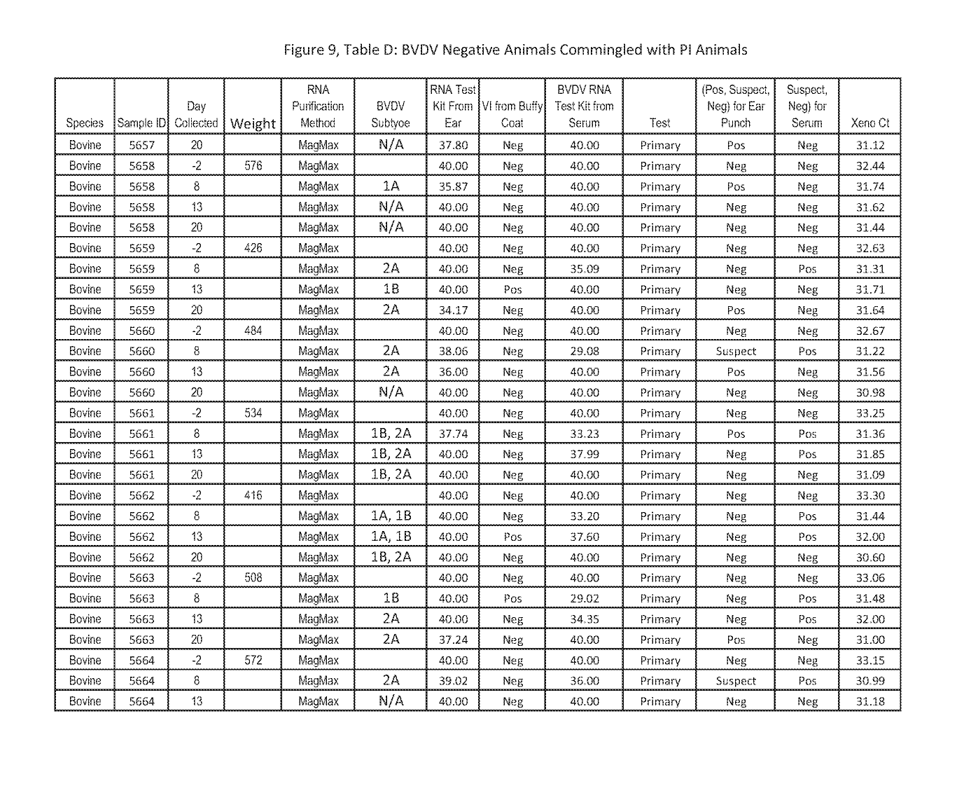

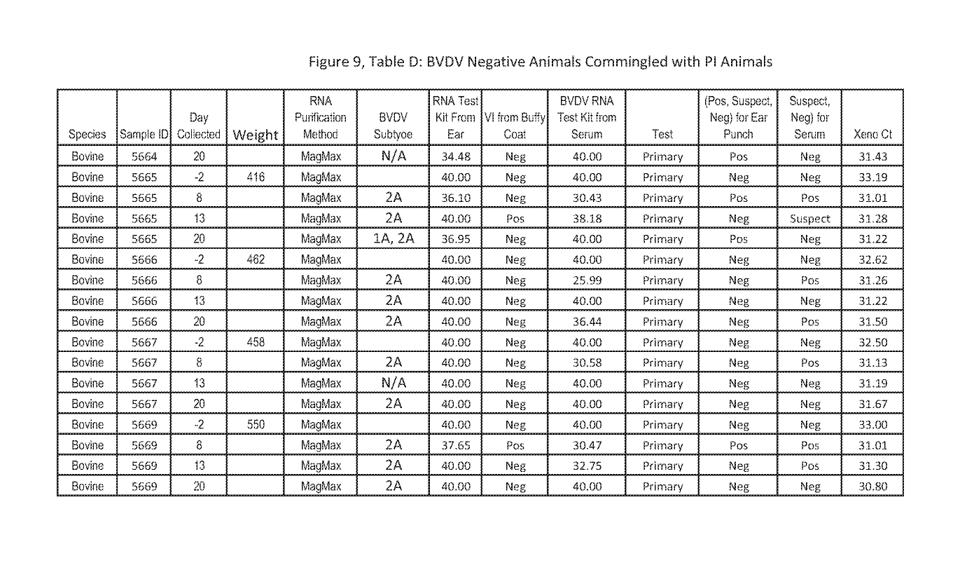

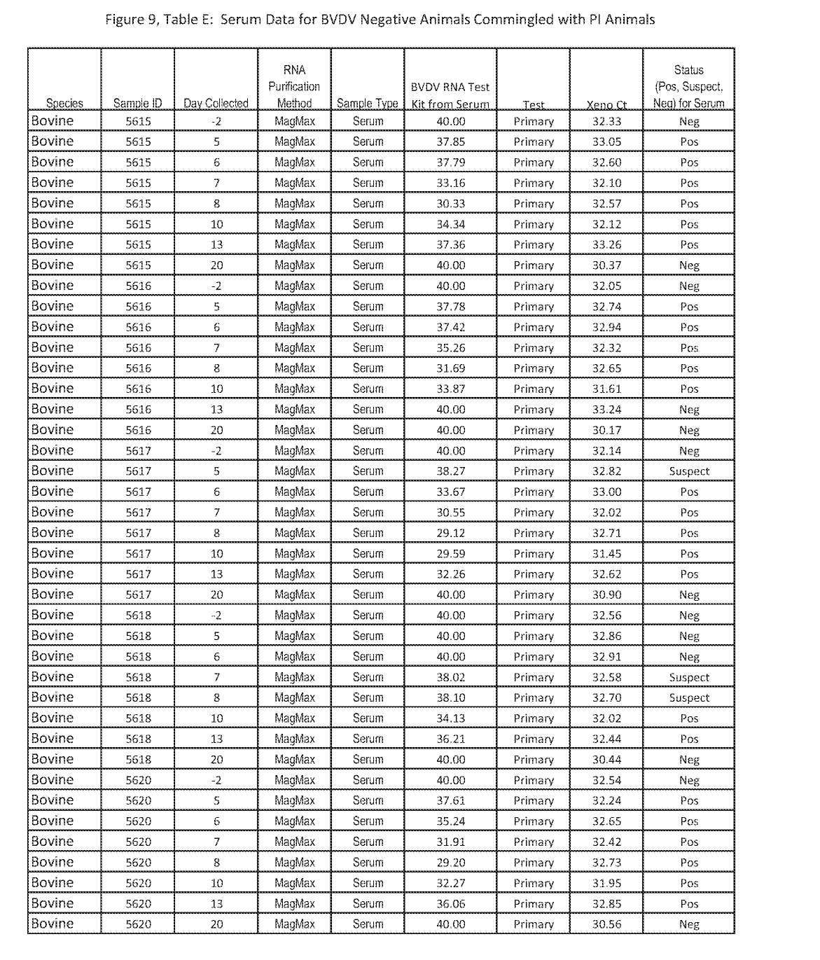

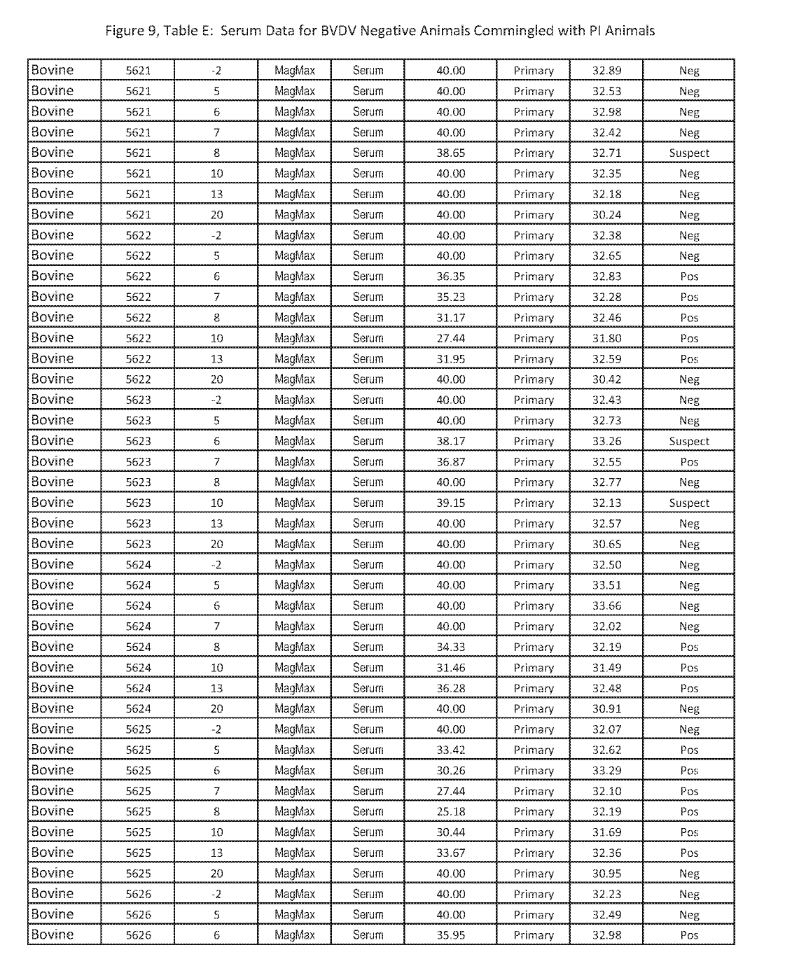

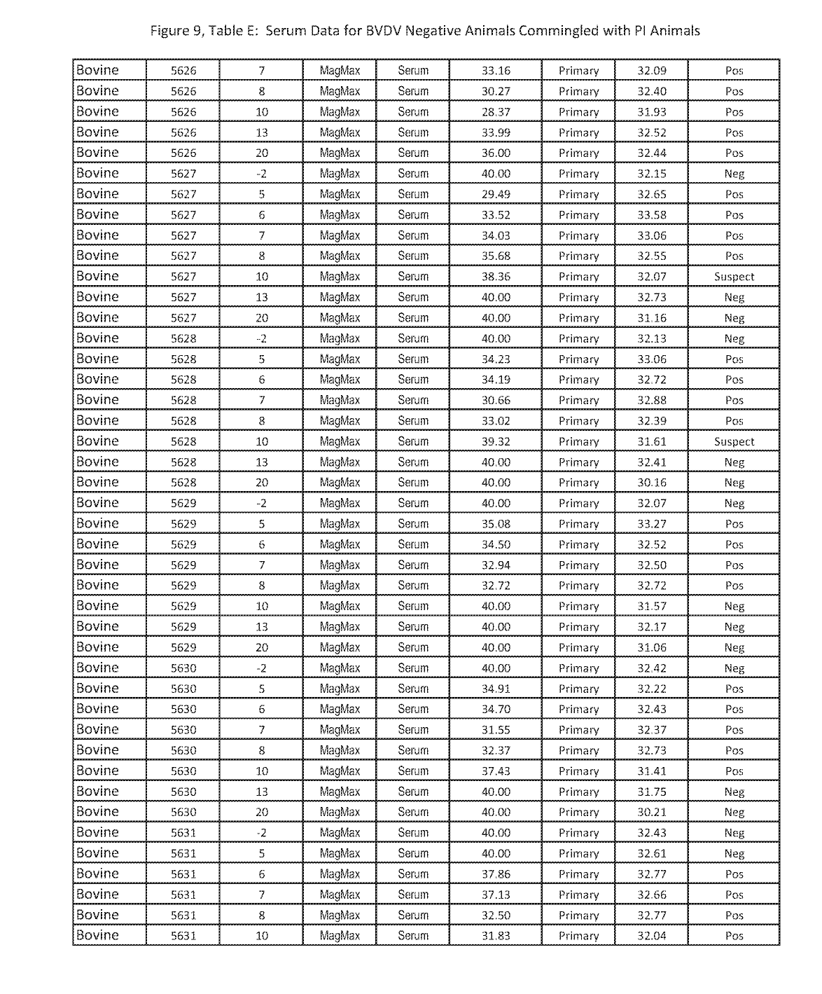

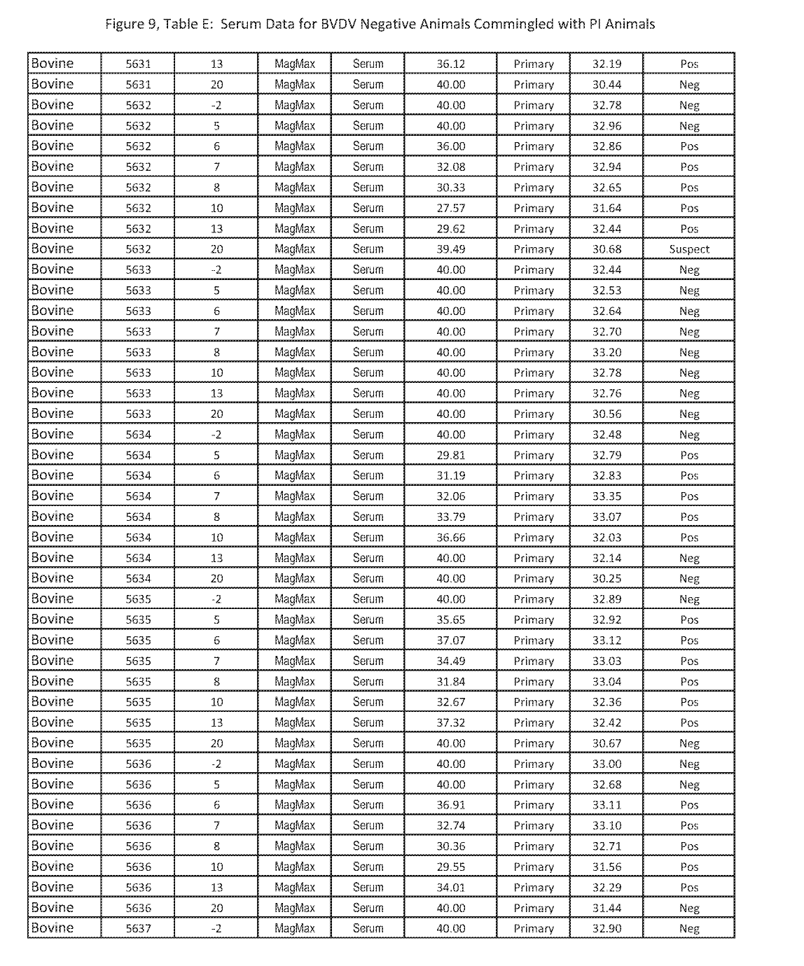

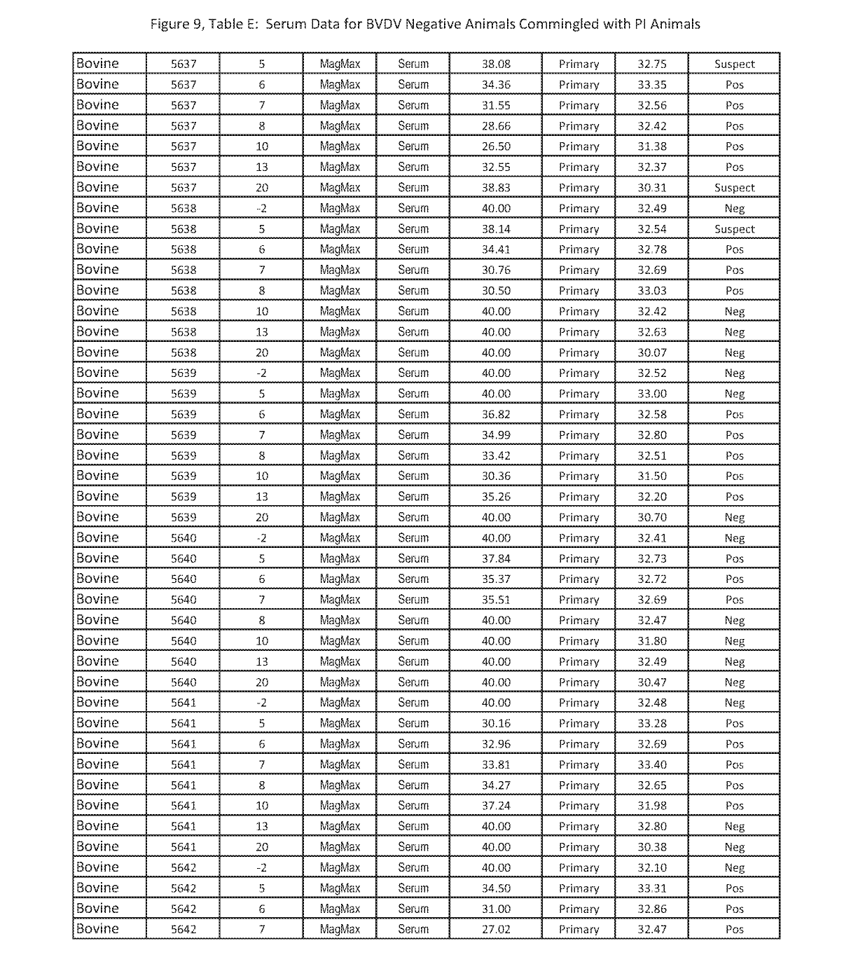

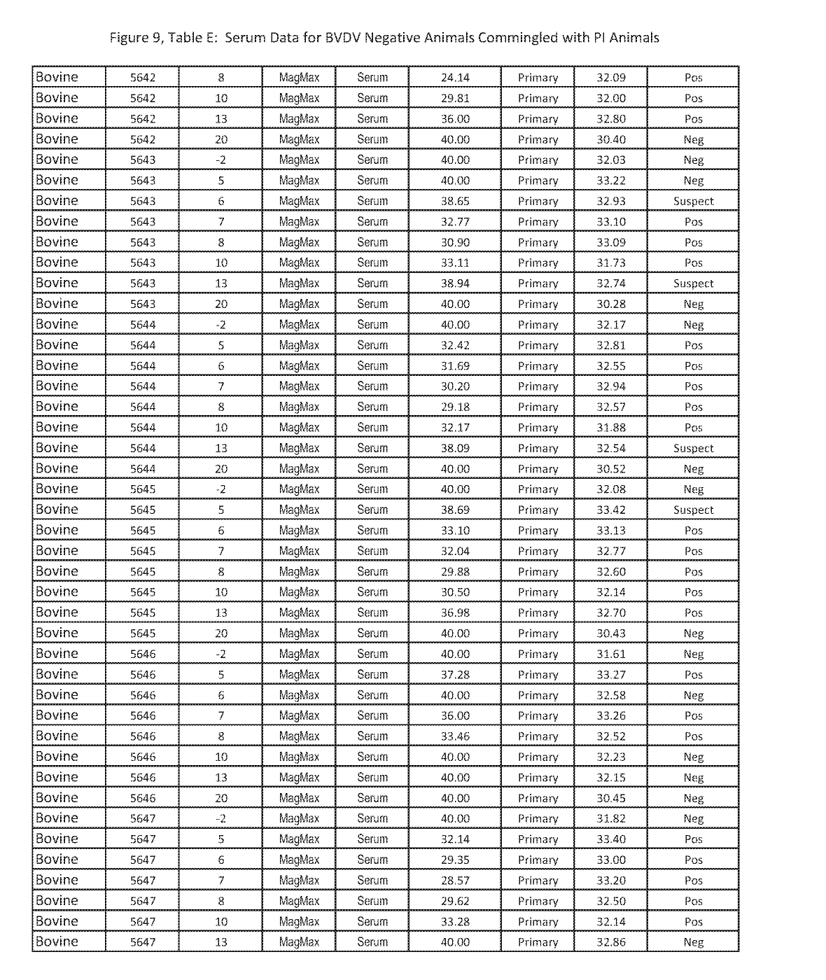

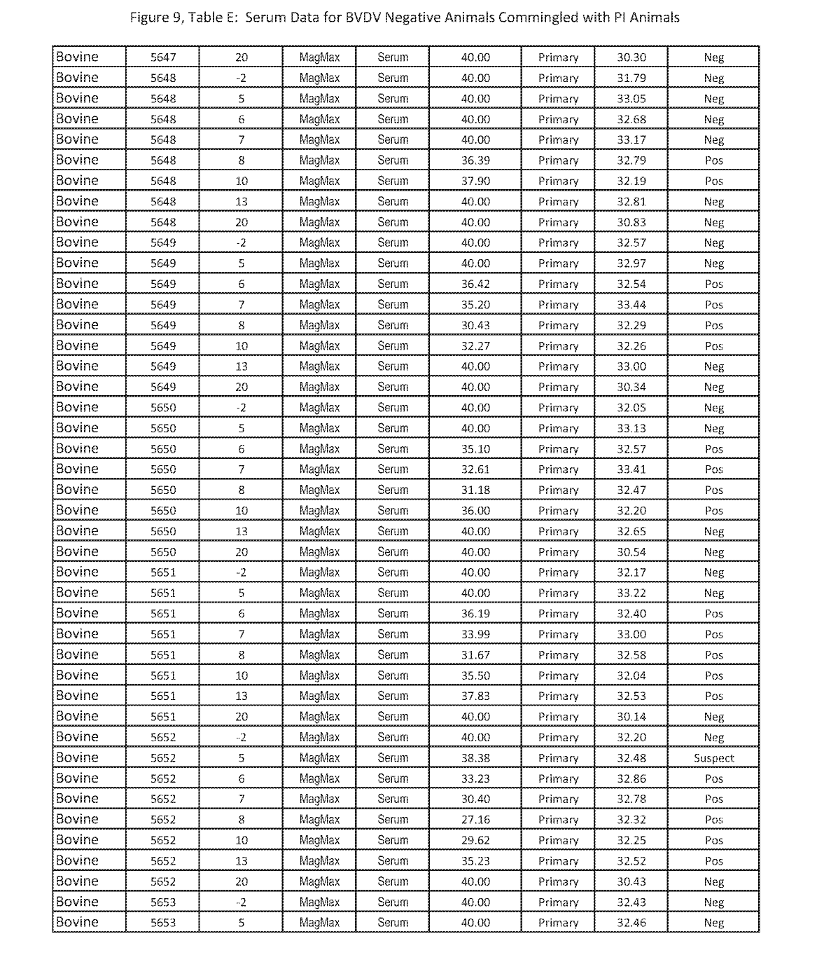

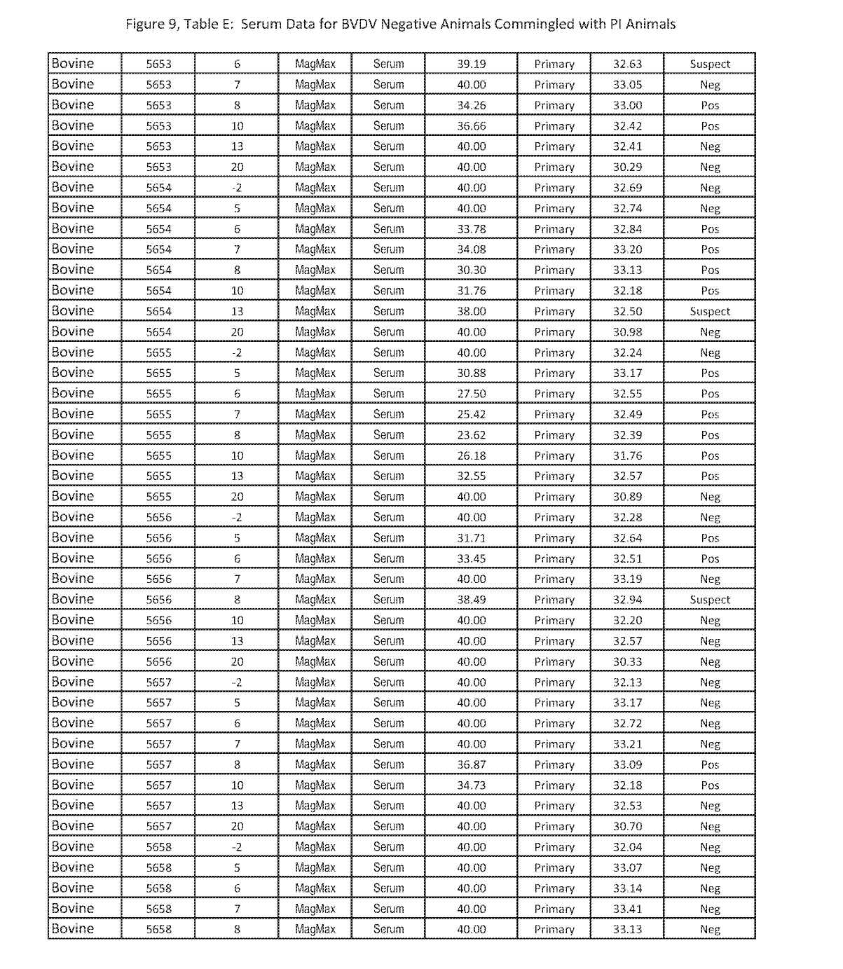

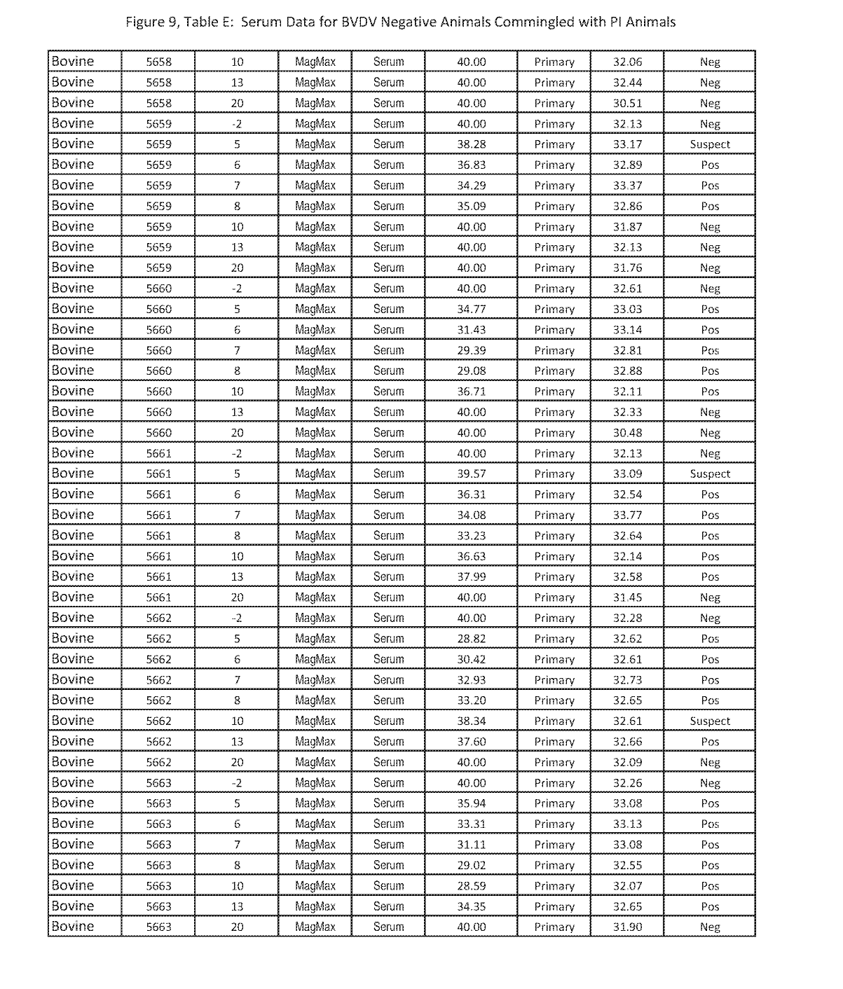

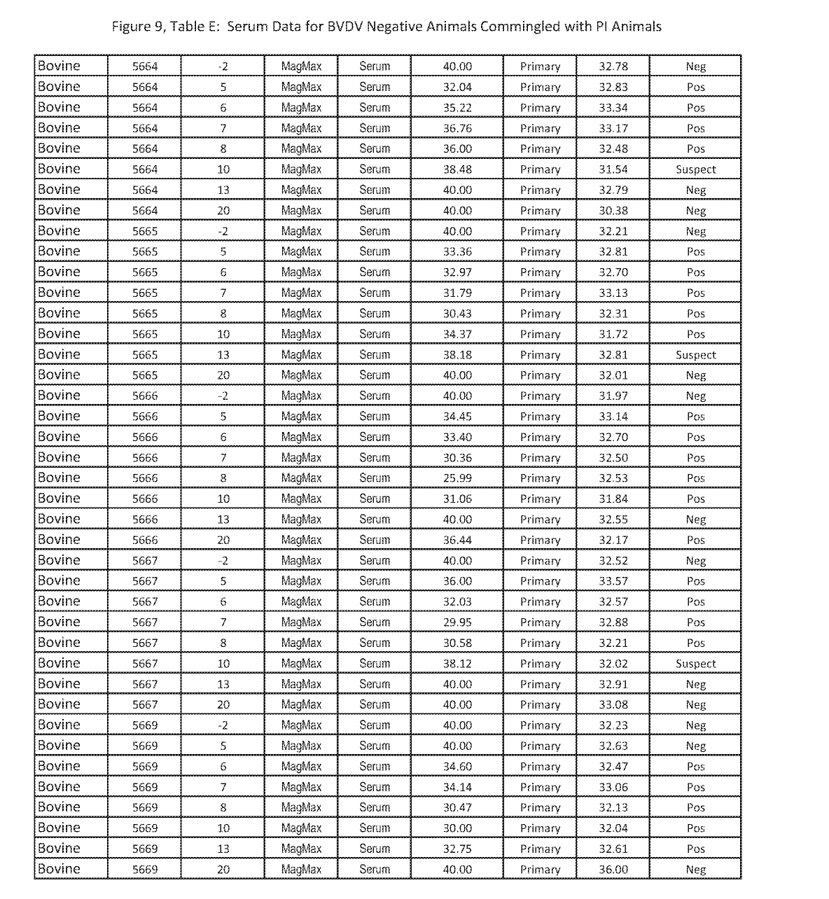

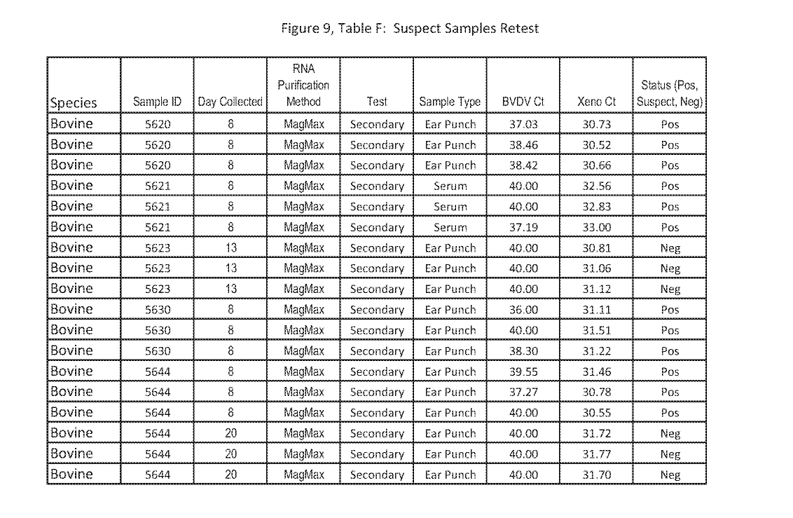

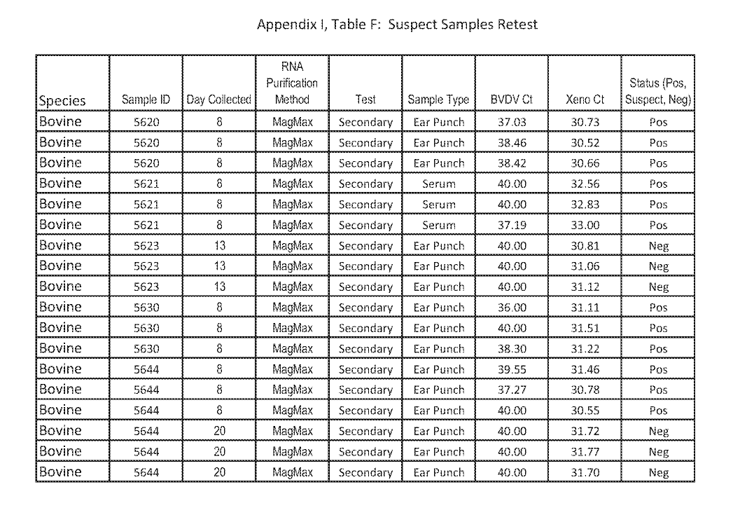

PI animals were identified by a veterinarian using antigen-capture ELISA (ACE). 71 screened animals were sampled from 5 different feedlots in Kansas. The animals included crossbred cattle between 6-12 months of age. The gender and weight of this group of animals was not recorded. Kansas is the number two state by number of cattle head and cattle per square mile in the United States. Sixteen of the top 25 cattle feeders have operations in Kansas. The usual sourcing for these operations includes, but is not limited to, animals originally from Kansas, Nebraska, Missouri, Iowa, and Oklahoma. The 71 PI animals were moved to holding pens at Kansas State University in Manhattan, Kans. Approximately 3 weeks after initial testing by the veterinarian, multiple sample types including blood (from which buffy coat and serum were collected), ear notches (3 mm punch), and nasal swabs were collected from each animal. At KSVDL, these samples were tested by several different diagnostic methods (ACE, VI, PCR, and IHC) to confirm PI status. Animals were only considered PI with positive results obtained from at least 2 of the above tests (Table 2). Sixty-three out of 71 animals met the PI characterization criteria (FIG. 9, Tables A-B).

BVDV subtypes were then determined by genotyping. Genotyping data obtained during secondary testing determined that 78% of the tested PI's were BVDV-1b genotype, 16% were BVDV-2a, and 6% were BVDV-1a. Ten apparently healthy PI animals were selected randomly from the larger group, based on the genotyping data, to represent their predominance in the field in a commingling study. All ten of the selected animals were females. The ten PI animals included two BVDV-1a, six BVDV-1b, and two BVDV-2a cattle. According to Ridpath et al. (J. Vet. Diag.—2010), the observed prevalence between BVDV types in 865 samples from South Midwestern feedlots were 12.1% for BVDV-1a, 75.3% for BVDV-1b, and 12.6% for BVDV-2a. In this study, among the 10 PI selected for the contact challenge, 20% of the PI animals were BVDV-1a, 60% were BVDV-1b, and 20% are BVDV-2a, which is an accurate representation of the variety of circulating types of strains. This is summarized in Table 3.

TABLE 3
PI Subtypes of Entire Study

| | South | | |
|---|---|---|---|
| | Midwestern US | 63 PI Study | 10 PI Subset |
| Subtype | (Ridpath paper) | Population | Population |
| 1A | 12% | 6% | 20% |
| 1B | 75% | 78% | 60% |
| 2A | 13% | 16% | 20% |

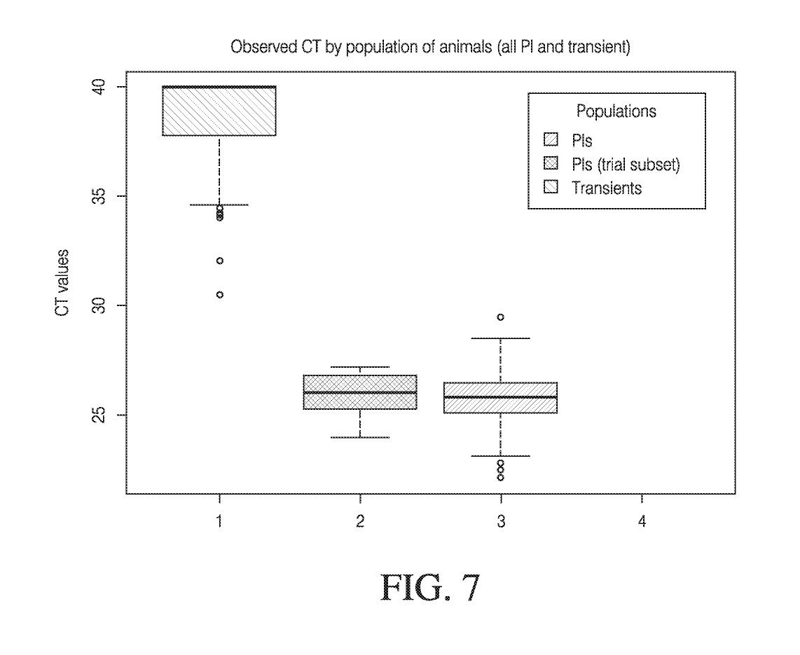

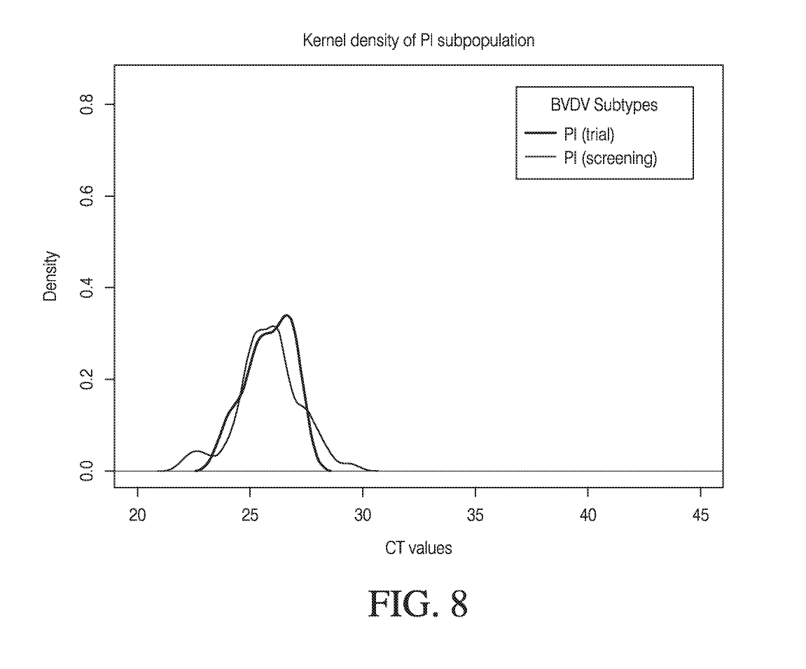

In addition to BVDV subtype, the 10 animals were representative of the larger subset of 63 PI animals in C_{T} range. Box plot and Kernel density estimators for the populations of interest in the study (screened PIs, PIs of the trial and contact animals or transients) are detailed in FIGS. 7-8. No statistical significance of distribution or average and dispersion were observed between the C_{T} observed for the 10 PI animals selected for the trial and the 63 screened from the field.

B. Recruitment and Characterization of BVDV Negative Animals for Commingling Study:

Fifty-three naïve cattle averaging 535 pounds (range: 416-742 pounds) were selected randomly through a broker from a sale barn/livestock market in Manhattan, Kans. The gender of each negative animal was not recorded. Specific geographical information for each animal prior to pen make up was unavailable. Vaccination history of all 53 naïve animals was unknown and additional husbandry information was not collected as this was an attempt to simulate a typical feedlot setting in the present transmission study. Prior to their inclusion in the commingling study, all the animals were tested for BVD status by ear notch, serum, and buffy coat PCR and determined not to be BVDV PI individuals.

C: Commingling Study:

The purpose of the commingling experiment was to create a controlled opportunity for BVDV-negative animals to become transiently infected with different types of BVDV. Samples were collected before and through the duration of the study for future testing using the BVD RNA Test Kit. Test data would be analyzed to determine if the distributions of C_{T} obtained from PI and TI animals were statistically different. This study was conducted at the Kansas State University Cow Calf Unit—Junietta. The 53 BVDV-negative cattle were allowed to commingle with the 10 PI animal subset in a single large holding pen for a period of 20 days. Study animals were provided with appropriate amounts of feed, water, and bunk space in accordance with standard feed lot practices. The cattle were moved around in the pen during the study to encourage commingling. The study animals were monitored for their performance and clinical signs, twice daily, throughout the study. Animals exhibiting respiratory signs (lethargy, cough, off feed, difficulty breathing, and depressed appearance) were pulled from the group and temperatures were taken. Animals exhibiting a temperature of 104° F. or higher were treated with a single 5 mL/100 lb dose of Baytril (Enrofloxacin) and returned to the study pen. For PI animals, samples were collected at day −2 and day 27 [7 days after study completion, as these animals had been previously characterized (FIG. 9, Table C)]. For the BVDV negative animals, blood samples were collected more frequently than ear punch samples due to animal welfare concerns raised by the KSU Institutional Animal Care and Use Committee (IACUC). Samples were collected for days −2 (2 days before commingling), 8, 13, and 20 for ear punch samples (FIG. 9, Table D) and days −2, 5, 6, 7, 8, 10, 13, and 20 for serum samples (FIG. 9, Table E).

D: Sample Nucleic Acid Purification:

Three millimeter punches were taken from the collected ear notches to standardize the sample size for analysis. BVDV nucleic acid purification was performed using the MagMAX™-96 Viral RNA Isolation Kit (AM 1836) and MagMAX™-Express instruments. Three millimeter ear notch punches or 50 μL of serum was used for BVDV nucleic acid purification. Eight thousand copies/reaction XenoRNA was spiked into the lysis/binding solution of each purification, and purified RNA was eluted in 90 μL of Elution Buffer.

E: Testing of Purified Nucleic Acid:

Purified RNA from all study samples were tested with the Bovine Virus Diarrhea RNA Test Kit (Lot 0910003) at Kansas State University Veterinary Diagnostic Laboratory (KSVDL) using the Bovine Diarrhea RNA Test Kit instructions for use, Rev. 01, 6/2009. 8 μL of purified BVDV RNA was used for each 25 μL PCR reaction. The reactions were performed on the AB 7500 Fast Real-Time PCR system using standard ramp mode and version 1.4 data analysis software.

Results:

Raw data to support all summarized results is presented in FIG. 9, Tables A-F. A statistical analysis summary is shown in Table 7. Detailed statistical calculations are not expressly shown here.

A. PI Cattle:

PI ear punch results: Primary testing resulted in the identification of 71 persistently infected (PI) cattle using Antigen Capture ELISA (ACE). Specimens were obtained by the testing lab and re-tested by ACE, IHC, and VI. Sixty-three animals were classified as PI after positive results were obtained from 2 of 3 confirmatory tests conducted (Table 2 and FIG. 9, Table A). Ear punch samples from all 71 animals were then tested with the BVD RNA Test Kit, data is summarized in Table 4. Data for the 8 cattle whose status were not confirmed by ACE, IHC, and VI and eliminated from the study can be found in FIG. 9, Table B.

TABLE 4
PI PCR test results

| | BVDV PCR Positive From |
|---|---|
| Cattle | Ear Punch |
| PI positive | 63 out of 63 |
| Unconfirmed status | 7 out of 8 |

B. Commingling Study:

For serum, of the 53 commingled BVDV-negative cattle, 50 animals tested positive for BVDV for at least one time point during the study after testing with the BVD RNA Test Kit, 2 were negative, and 1 resulted in a negative or suspect result (FIG. 9, Table D). For the ear punch samples collected on days 8, 13, and 20, eleven animals tested negative throughout the study, 10 of which were positive by serum analysis, and 1 suspect. Four additional animals had a suspect result on at least one of the three days tested and negative for the other days. In summary, primary testing of the 53 animals used in the PI commingling study produced 38 positive, 11 negative and 4 “suspect” animals from the ear punch data.

The samples that met the criteria for “suspect” were retested according to the suspect workflow provided in the instructions for use. The suspect workflow called for each sample to be retested in triplicate using the original purified RNA. If 1 out of the three replicates were positive, then the final test result was presumptive positive. If ≧2 out of the three replicates were positive, then the final Results are considered positive if a C_{T} value of less than 38 is achieved for any of the collection time points. A sample is considered negative if no time points had a C_{T} below 40 signifying no signal. An animal is considered “suspect” if a signal between C_{T} 38 and 40 is achieved at any time point where all of the other days are negative. The suspect workflow called for each sample to be retested in triplicate. Summarized results for investigation of suspect samples is detailed in Table 5 below. Of the four suspect ear punch samples, three were characterized as positive after the suspect workflow. Suspect sample raw data is located in FIG. 9, Table F.

TABLE 5
Status of suspect results after retest

| | | | | | Status (Pos, |
|---|---|---|---|---|---|
| Sample | Day | | Sample | Number | Suspect, |
| ID | Collected | Test | Type | Positive | Neg) |
| 5620 | 8 | Secondary | Ear Punch | 3 out of 3 | Pos |
| 5621 | 8 | Secondary | Serum | 1 out of 3 | Pos |
| 5623 | 13 | Secondary | Ear Punch | 0 out of 3 | Neg |
| 5630 | 8 | Secondary | Ear Punch | 2 out of 3 | Pos |
| 5644 | 8 | Secondary | Ear Punch | 2 out of 3 | Pos |
| 5644 | 20 | Secondary | Ear Punch | 0 out of 3 | Neg |

A summary of the commingling study is shown in Table 6 below. After commingling with 10 PI animals, 51 of the 53 confirmed negative animals tested positive by PCR analysis of the serum; 41 of which were positive by PCR analysis of the ear punches on 1 or more days of collection, after the suspect work flow was followed. The late C_{T}s for the transiently infected animals were statistically different that the C_{T}s observed for persistently infected animals. See FIG. 9 and Tables A and C therein for raw C_{T} values. Summary of statistical calculations are in Table 7 below.

TABLE 6
Summary results from co-mingling study

| | BVDV PCR | BVDV PCR |
|---|---|---|
| | Positive From | Positive From Ear |
| Cattle | Serum | Punch |
| PI animals | 10 out of 10 | 10 out of 10 |
| Co-mingled animals | 51 out of 53 | 41 out of 53 |

C. Statistical Analysis:

A detailed statistical analysis was performed. There were 4 subpopulations from the total study population identified: (1) PI selected and used in the contact trial, tested at 27 days after the trial and −2 days; (2) PI screened and confirmed (screening ACE, confirmation with at least two positive result among ACE, IHC and VI); (3) Negative animals place in contact and tested at −2, 8, 13 and 20 days.

The fourth subpopulation represented suspected PI animals that were not confirmed with at least two positive result among ACE, IHC and VI) and therefore excluded of the PI population.

TABLE 7
Summary of statistics of each population

| Population | n = | missing | unique | Mean | Min | 1st Q. | Median | 3rd Q. | Max |
|---|---|---|---|---|---|---|---|---|---|
| PI Subset used in | 20 | 0 | 20 | 25.86 | 23.93 | 25.32 | 26.01 | 26.75 | 27.17 |
| Contact Trial (1) | | | | | | | | | |
| All Screened PI's | 63 | 0 | 63 | 25.84 | 22.11 | 25.09 | 25.82 | 26.47 | 29.49 |
| (2) | | | | | | | | | |
| Negative Contact | 212 | 0 | 65 | 38.95 | 30.51 | 37.81 | 40.00 | 40.00 | 40.00 |
| Animals (3) | | | | | | | | | |

The distribution (minimum, maximum, median and quartiles) and summary statistics (mean) are really similar for the two populations of PI animals. More than half the CTs observed in the population of the negative animals that have been put in contact with the PIs are equal to 40.

The graph in FIG. 6 represents the distribution as a histogram of the 3 populations of interest. It confirms what appeared in the previous summary statistics that two populations, namely the PI population selected for the trial and the PI population screened and confirmed from the field seems to originate from the same population. In addition, the PI population and transient population are distinguishable according to Ct distribution. No overlap of PI and TI Ct's between 29.49 and 30.51 was seen (FIG. 6).

The graphs presented in FIGS. 7-8 compare the distributions of the populations of interest enrolled in the study. FIG. 7 is a box plot. FIG. 8 show the kernel smoothed density. Both graphs seem to indicate that the two populations (All PIs and PI trial subset) are sampled from the same population. Furthermore, the two populations of PI animal are suggestive of a Gaussian distribution. In contrast, the negative animals enrolled, and put in contact with the PI at the beginning of the trial, present a distribution that is right censored at 40 CT, skewed to the lower value.

Additional statistical analyses were also performed on these results to determine: 1) Effect of day of sampling on the PI subpopulation used for the trial; 2) Effect of virus type on the PI subpopulations; 3) Comparison of the two PI populations—screening and the PI subpopulation used for the trial; 4) Normality assumption; 5) Discussion about the representivity of the “positive” population; 6) Effect of day after challenge on the negative population; 7) Effect of virus type on the negative population; 8) Non normality of the population; 9) Receiver Operating Characteristic (ROC) Analysis. These detailed reports are not expressly described herein.

Each embodiment disclosed herein may be used or otherwise combined with any of the other embodiments disclosed. Any element of any embodiment may be used in any embodiment. Although the dislcosure has been described with reference to specific embodiments, it will be understood by those skilled in the art that various changes may be made and equivalents may be substituted for elements thereof without departing from the true spirit and scope of the dislcosure. In addition, modification may be made without departing from the essential teachings of the dislcosure.

What is claimed is:

1. A method for diagnosing an animal as persistently infected (PI) with a bovine viral diarrhea virus (BVDV) comprising:
a) performing a single BVDV polymerase chain reaction (PCR) test comprising:
1) contacting nucleic acids from a sample derived from an animal with at least one primer set that is specific to hybridize to one or more BVDV-specific target nucleic acids;
2) performing an PCR amplification on nucleic acids from the sample hybridized to the at least one primer set; and
3) detecting one or more BVDV-specific amplification products; and
b) determining Ct value of the BVDV-specific amplification product, wherein a Ct value of ≦31 indicates that the animal is a PI animal.

2. The method of claim 1, wherein a Ct value in the range of from about 20 about 31 indicates an animal is a PI animal.

3. The method of claim 1, wherein the nucleic acids from the sample comprise DNA, RNA, genomic DNA and combinations thereof.

4. The method of claim 1, wherein the nucleic acids from the sample comprise BVDV RNA molecules and the method further comprises performing an reverse transcriptase reaction on nucleic acids from the sample prior to performing the PCR test.

5. The method of claim 1, wherein the BVDV virus is a BVDV subtype 1A virus, a BVDV subtype 1B virus or a BVDV subtype 2A virus.

6. The method of claim 1, wherein determining Ct value comprises calculating the BVDV titer.

7. The method of claim 1, wherein the sample derived from the animal is an ear notch sample, a hair sample, a tail hair sample, a serum sample, a blood sample, a milk sample, a urine sample, a fecal sample, a skin sample, a lymph sample, a plasma sample, a cerebrospinal fluid sample, a mucus sample, a throat swab sample, or any sample from a bodily fluid or tissue of an animal suspected of having BVDV.

8. A method for diagnosing an animal as transiently infected (TI) with a bovine viral diarrhea virus (BVDV) comprising:
a) performing a single BVDV polymerase chain reaction (PCR) test comprising:
1) contacting nucleic acids from a sample derived from an animal with at least one primer set that is specific to hybridize to one or more BVDV-specific target nucleic acids;
2) performing an PCR amplification on nucleic acids from the sample hybridized to the at least one primer set; and
3) detecting one or more BVDV-specific amplification products; and
b) determining Ct value of the BVDV-specific amplification product, wherein a Ct value of >31 indicates that the animal is a TI animal.

9. The method of claim 8, wherein the Ct value is a range of values of from about 32 to about 40.

10. The method of claim 8, wherein the BVDV virus is a BVDV subtype 1A virus, a BVDV subtype 1B virus or a BVDV subtype 2A virus.

11. The method of claim 8, wherein determining Ct value comprises calculating the BVDV titer.

12. The method of claim 8, wherein the sample derived from the animal is an ear notch sample, a hair sample, a tail hair sample, a serum sample, a blood sample, a milk sample, a urine sample, a fecal sample, a skin sample, a lymph sample, a plasma sample, a cerebrospinal fluid sample, a mucus sample, a throat swab sample, or any sample from a bodily fluid or tissue of an animal suspected of having BVDV.

13. A method to distinguish an animal with a BVDV infection as a PI animal or a TI animal comprising:
performing a PCR reaction to amplify and detect the presence of one or more BVDV-specific target nucleic acids in a sample obtained from the animal; and
determining Ct value of one or more BVDV-specific target nucleic acid amplification products, wherein a Ct value of >31 indicates that the animal is a TI animal and a Ct value ≦31 indicates that the animal is a PI animal.

14. The method of claim 13, wherein at least one BVDV-specific target nucleic acid in the sample is an RNA and the method further comprises performing an reverse transcriptase (RT) reaction to convert the BVDV-specific RNA molecule into a cDNA molecule prior to performing the PCR reaction.

15. The method of claim 13, wherein a Ct value in the range of from about 32 to about 40 is indicative of TI in the animal, and a Ct value in the range of from about 20 to about 31 is indicative of PI in the animal.

16. The method of claim 13, wherein the BVDV virus is a BVDV subtype 1A virus, a BVDV subtype 1B virus or a BVDV subtype 2A virus.

17. The method of claim 13, wherein determining Ct value comprises calculating the BVDV titer.

18. The method of claim 13, wherein the sample obtained from the animal is an ear notch sample, a hair sample, a tail hair sample, a serum sample, a blood sample, a milk sample, a urine sample, a fecal sample, a skin sample, a lymph sample, a plasma sample, a cerebrospinal fluid sample, a mucus sample, a throat swab sample, or any sample from a bodily fluid or tissue from an animal suspected of having BVDV.

19. The method of claim 13, wherein the animal is a mammal, a cattle, a sheep, a goat, a deer or an alpaca.
